SEQUENCE LISTING (2 sequences)

SEQ ID NO: 1 - DNA, 21 bp, Artificial Sequence
  Oligonucleotide primer to amplify hIL-6
ctcgagatgaactccttctcc

SEQ ID NO: 2 - DNA, 21 bp, Artificial Sequence
  Oligonucleotide primer to amplify hIL-6
tacgtactacatttgccgaag

(19) United States
(12) Patent Application Publication
(10) Pub. No.: US 2003/0040498 A1
(43) Pub. Date: Feb. 27, 2003

(54) Oncolytic RNA replicons

(76) Inventors: David Calvert Ansardi, Warrior, AL (US); Casey Dolan Morrow, Birmingham, AL (US); Donna Coker Porter, Warrior, AL (US)

Correspondence Address: BAKER & BOTTS, 30 ROCKEFELLER PLAZA, NEW YORK, NY, 10112

(21) Appl. No.: 10/097,058

(22) Filed: Mar. 13, 2002

Related U.S. Application Data
(60) Provisional application No. 60/275,840, filed on Mar. 14, 2001.

(51) Int. Cl. A61K 48/00

(52) U.S. Cl. 514/44; 424/93.2

(57) ABSTRACT

The limited efficacy and/or toxicity of conventional therapies for many types of human cancers underscores the need for development of safe and effective alternative treatments. Towards this goal, the invention describes the direct oncolytic activity of RNA-based vectors derived from poliovirus, termed replicons, which are genetically incapable of producing infectious virus. Replicons of the invention are cytopathic in vitro for human tumor cells originating from brain, breast, lung, ovaries and skin (melanoma). Injection of replicons into established xenograft flank tumors in scid mice resulted in oncolytic activity and extended survival. Inoculation of replicons into established intracranial xenografts tumors in scid mice resulted in tumor infection and extended survival. Histological analysis revealed that replicons infected tumor cells at the site of inoculation and, most importantly, diffused to infect tumor cells which had metastasized from the initial site of implantation. The wide spectrum of cytopathic activity for human tumors combined with effective distribution following in vivo inoculation establishes the therapeutic potential of poliovirus replicons for a variety of cancers.

### SPECIFICATION

This application is based on U.S. Provisional Application No. 60/275,840, filed Mar. 14, 2001, which is incorporated herein in its entirety by reference.

This invention was made with government support under National Institutes of Health/National Cancer Institute SBIR Phase I grants 1R43CA79355-01 and 1R43CA83616-01 awarded to Donna C. Porter and David C. Ansardi respectively. The government has certain rights in the invention.

### BACKGROUND OF THE INVENTION

Many malignant tumors respond poorly to current methods of treatment such as surgical resection, radiation therapy, and chemotherapy, with such methods often producing significant side effects. Consequently, treatments with greater efficacy, but with fewer and less severe side effects, must be sought. The use of viruses for the treatment of cancer has been investigated for almost fifty years (Asano T, 1974, Cancer 34:1907-1928; Moore AE, “Viruses with oncolytic properties and their adaptation to tumors”, Annals of New York Acd. of Sci, pp. 945-952; Moore J P et al., 1993, J. Virol. 67:863-875; Southam C M, 1960, “Present status of oncolytic virus studies”, The New York Academy of Sciences, pp. 657-673; Taylor M W et al., 1971, Pro. Natl. Acad. Sci. USA 68:836-840). Early on, viruses were identified which could selectively kill tumor cells without killing normal non-neoplastic cells. Work with the paramyxovirus, New Castle Disease Virus, showed promise in clinical trials as an anti-neoplastic agent (Cassel W A et al., 1963, Cancer 18:863-868; Cassel W A et al., 1983, Cancer 52:856-860; Lorence R M et al., 1994, Cancer Res. 54:6017-6021; Lorence R M et al., 1994, J. Natl. Can. Inst. 86:1228-1233; Reichard K W et al., 1992, J. of Surg. Res. 52:448-453; Smith R R et al., 1956, Cancer 9:1211-1218). Even with apparent neoplastic cell specific infection though, a concern still existed with respect to reversion for growth in non-neoplastic cells. The advent of molecular biology allowed the capacity for genetic manipulation of adenovirus, herpesvirus (HSV), or proviral genomes of retroviruses, to be engineered so as to allow a single round of infection without spread to neighboring cells (Roth J A et al., 1997, J. Natl. Can. Inst. 89:21-39). Subsequently, viruses have been generated to selectively replicate in tumor, but not normal cells by virtue of a viral dependence on a tumor specific protein (Khuri F R et al., 2000, Nature Med. 6:879-885; Strong J E et al., 1998, EMBO J. 17:3351-3362). Viruses have also been engineered to encode a cytotoxic protein to express a “suicide gene” that operates in conjunction with a prodrug (Klatzmann D et al., 1996, Human Gene Therapy 7:109-126; Andreansky S S et al., 1996, Proc. Natl. Acad. Sci. USA 93:11313-11318; Andreansky S et al., 1997, Cancer Research 57:1502-1509; Hughes B W et al., 1995, Cancer Research 55:3339-3345; Mullen C A et al., 1992, Proc. Natl. Acad. Sci. USA 89:33-37; Mullen C A et al., 1994, Cancer Research 54:1503-1506). This requirement introduces more complexity into the treatment system, and the potential toxicity of the prodrug or its toxic metabolite for normal tissues also must be considered. Even with these advancements in genetic engineering of viruses, a delicate balance is maintained between the capacity to selectively kill tumor cells and potential for pathogenicity in the host that has lead to the failure of clinical trials.

The potential problems associated with many of viral vectors underscores the need for additional advancements. This is particularly true of brain tumors, such as glioblastomas. Malignant gliomas have proven to be a very difficult cancer to control and have resisted the various therapeutic interventions that have been attempted to treat this devastating disease. Most patients diagnosed with glioblastoma multiforme undergo surgical intervention in conjunction with radiation therapy and/or chemotherapeutic treatments. Nevertheless, despite these aggressive approaches to therapy, most patients die within one year of diagnosis (Kim J H et al., 1994, Cancer Res. 54:6053-6055; Andreansky S et al., 1996, Proc. Natl. Acad. Sci. USA 93:11313-11318). These survival odds have changed very little during the last thirty years, despite advances in imaging and detection, surgical techniques, chemotherapy and radiation therapy (Komblith P K et al.,1993, Surg. Neurol. 39:538-543). The poor prognosis for glioma patients and the resistance of the disease to traditional therapies underscore the necessity for development of effective treatments.

In recent years, new DNA-delivery/gene therapy-based strategies have been proposed to treat glioma. Most of these employ vectors derived from modification of DNA viruses, such as adenovirus or herpesvirus or vectors derived from retroviruses. DNA viral vectors may either be replication competent or limited to a single round of “infection” and unable to spread to neighboring cells. Typically these vectors contain a transgene which upon expression produces a cytotoxic protein or encodes a “suicide gene” which upon expression operates in conjunction with a prodrug. In a few cases, non-pathogenic, replication-competent variants of viruses such as HSV-1 have been tested for direct tumor cytotoxicity. Some of these DNA delivery/gene therapy strategies have been or are being tested in Phase 1 of clinical studies of glioma patients (Klatzmann D et al., 1996, Human Gene Therapy, 7:109-126).

Despite the promise of new treatments for malignant gliomas and other cancers using viral-based and other gene therapy vectors, the limitations and pitfalls of many of these systems highlight the need for additional exploration and development in this area. For example, use of retroviral and some DNA-based viral vectors exposes patients to the risk of integration of recombinant sequences into human chromosomal DNA. Such insertion events may be mutagenic and, if so, may lead to tumor formation if critical genes are activated or suppressed. Treatment with a fully replication competent virus risks the pathological consequences that may occur if the virus reverts to a virulent form. Finally, expression of proteins encoded by DNA-based vector systems depends on the efficiency of vector uptake into host cells, transport to the nucleus, and promoter activation. This dependency may result in a significant lag time in onset of foreign protein accumulation and reduce the amount of protein produced. Recent studies have also suggested that long term expression of foreign genes can lead to an inflammatory response that may, in turn, result in unwanted pathogenesis (Barba D et al., 1994, Proc. Natl. Acad. Sci. USA 91:4348-4352 1994).

A new approach to the development of anti-cancer vectors would be to develop small RNA viruses as an alternative to the current DNA systems. RNA-based vector systems are not susceptible to integration into host cell chromosomes and reduce the potential for side effects due to long term gene expression. Additionally, RNA viral vectors enable more rapid and higher levels of gene expression, due, in part, to inhibition of host cell protein synthesis.

Poliovirus, a small RNA-virus of the family Picornaviridae, is an attractive candidate system for treatment of glioma and other cancers because of several biological features inherent to the virus. First, the live attenuated strains of poliovirus are safe for humans and are routinely administered to the general population in the form of the Sabin oral vaccine. A viral genome adapted for use in cancer therapy, therefore, should pose no greater health risk than that associated with administration of the attenuated vaccines alone.

Second, the pathogenesis of the virus is well studied and important features have been identified. Poliovirus is transmitted by an oral-fecal route and is stable in the harsh conditions of the intestinal tract. Primary replication occurs in the oropharynx and gastrointestinal tract, with subsequent spread to the lymph nodes (Horstmann, D M et al., 1959, JAMA 170:1-8). The virus exhibits a restricted cell tropism in vivo confined to mainly the anterior horn cells of the central nervous system.

Upon entry into host cells, the RNA genome undergoes a rapid amplification cycle followed by an intense period of viral protein production. During this period, a poliovirus-encoded 2A protease arrests host cell cap-dependent protein synthesis by cleaving eukaryotic translation initiation factor 4GI (eIF4GI) and/or eIF4GII (Goldstaub D et al., 2000, Mol. Cell Biol. 20(4):1271-1277). Host cell protein synthesis may also be inhibited by proteolytic inactivation of transcription factors required for host cell gene expression (Das S et al., 1993, J. Virol. 67:3326-3331). The arrest of host cell protein synthesis allows poliovirus RNA, which does not require a 5′ cap for translation, to be selectively expressed over host transcripts. Moreover, arrested host cell protein synthesis is detrimental to the cell and ultimately contributes to its death.

Third, the entire poliovirus genome has been cloned and sequenced and the viral proteins identified. An infectious poliovirus cDNA is also available which has allowed further genetic manipulation of the virus (Racaniello V R et al., 1981 Science 214(4542) 916-919). Poliovirus contains a single-stranded RNA genome of approximately 7500 bases in length. The viral RNA genome encodes the necessary proteins required for generation of new progeny RNA, as well as encapsidation of the new RNA genomes. In vitro, poliovirus is lytic, resulting in the complete destruction of permissive cells. Since the viral replication cycle does not include any DNA intermediates, there is no possibility of integration of viral DNA into the host chromosomal DNA.

Fourth, the human poliovirus receptor (HPVR; CD155) has been cloned (Mendelsohn C L et al., 1989, Cell 56:855-865) and characterized. hPVR, a member of the immunoglobulin superfamily (Mendelsohn C L et al., 1989, Cell 56:855-865), is a three domain, surface glycoprotein and is required for uncoating the viral genome upon infection (Bernhardt G et al., 1994, “The poliovirus receptor: identification of domains and amino acid residues critical for virus binding”, Virology 203:344-356). hPVR has been found on many human cell types, including the anterior horn cells of the central nervous system and various cancer cells, such as malignant gliomas. However, hPVR expression alone may not be sufficient to direct poliovirus' tropism, since poliovirus-infected transgenic mice which express the hPVR on all cells still show restricted tropism (Ren et al., 1992, J. Virol. 66:296-304).

Recent studies have demonstrated expression of CD 155 on a number of human cancer cell lines of various origins, including epidermoid carcinoma, breast carcinoma, osteocarcinoma, neuroblastoma and glioblastoma (Solecki D et al., 2000, J. Biol. Chem. 275:12453-12462; Solecki D et al., 1999, J. Biol. Chem. 274:1791-1800). Expression of CD155 has also been reported to occur on a high percentage of patient CNS tumors of glial cell origin (astrocytoma, oligodendroglioma, and glioblastoma multiforme) (Solecki D et al., 2000, J. Biol. Chem. 275:12453-12462; Solecki D et al., 1999, J. Biol. Chem. 274:1791-1800; Gromeier M et al., 2000, Proc. Natl. Acad. Sci. USA 97(12):6803-6808). In contrast, previous studies have found the expression of CD 155 to be virtually undetectable in normal, non transformed cells (Gromeier M et al., 2000, Virol. 273:248-257). This could be due to the fact that the promoter for the receptor is active only during a short time of development (Id.). The preferential expression of CD 155 on tumor, but not the normal cells, suggests that CD155 could be a unique tumor marker (Solecki D et al., 2000, J. Biol. Chem. 275:12453-12462; Solecki D et al., 1999, J. Biol. Chem. 274:1791-1800).

Fifth, poliovirus may trigger oncolysis. The first indication that poliovirus possesses oncolytic properties came from researchers in the former Soviet Union, who discovered that wild type poliovirus stimulated oncolysis in short term organ cultures of gastrointestinal tract tumor explants (Tsypkin L B et al., 1976, Cancer 38:1796-1806). However, the work was limited to infection of cultures of tumor explants with replication competent pathogenic virus (Voroshilova M K et al., 1970, in Enterovirus infections: transactions of the institute ofpoliomyelitis and virus encephalitides, Vol XIV, Chumakov ed., Moscow, pp.339-340; Voroshilova M K et al., 1974, Acta Virol. 18:129-134; Tsypkin L B et al., 1976, Cancer 38:1796-1806) and no therapeutic application was proposed. More recently, after finding that the presence of a CD155 antigen correlated with susceptibility to virus induced cell lysis, Gromeier et al. have proposed using a replication-competent poliovirus in glioma therapy (2000, PNAS 97:6803-6808). The attenuated human poliovirus used contained an internal ribosome entry site (IRES) element from the related human rhinovirus substituted for the corresponding element in poliovirus. These viruses are capable of replication and cell-to-cell spread, although they appear to be incapable of causing neuropathogenesis (2000, PNAS 97:6803-6808).

However, this approach is encumbered by several drawbacks. First, the ability of these IRES vectors to self propagate may enable them to spread beyond the targeted cells in an uncontrolled manner. This would be a particular concern in the case of a recombinant viral vector derived from IRES vectors containing a transgene that encodes a toxic molecule. A second concern is that sustained expression of the transgene may lead to unintended and undesirable effects. Finally, upon multiple rounds of replication and infection in vivo, the virus may mutate and regain the capacity for causing neuropathogenesis, which has happened on rare occasions when using oral polio vaccines.

In view of the compelling need for effective treatments and the limitations of existing technology, it is desirable to develop improved compositions and methods for cancer therapy. Such improved compositions and methods may be safer or more effective than present methods. Such improved compositions and methods may also reduce the number or severity of side effects associated with present practices.

### SUMMARY OF THE INVENTION

The present invention relates to poliovirus-based replicons which possess oncolytic activity towards the cells of at least one type of tumor. Replicons of the invention lack at least a portion of a sequence necessary for encapsidation and cannot produce new encapsidated vectors following entry into a cell. However, replicons of the invention are fully capable of RNA replication (amplification) upon introduction into cells and may comprise translatable sequences.

The present invention further relates to methods of using poliovirus-based replicons to kill tumor cells, which in turn provides a new means for carrying out cancer therapy. The methods involve (i) optional administration of one or more agents which increase the amount of poliovirus receptor present in target cells and (ii) contact of such target cells with oncolytic replicons, in a manner such that the replicons are taken up by the target cells and cause lysis thereof. Replicons may or may not contain non-poliovirus sequences. In some embodiments of the invention, the method further comprises contacting encapsidated oncolytic replicons with CD 155 on the surface of the target cells.

### BRIEF DESCRIPTION OF THE DRAWINGS

The patent application file contains at least one drawing executed in color. Copies of this patent or patent application publication with color drawing(s) will be provided by the United States Patent and Trademark Office upon request and payment of the necessary fee.

FIG. 1. Diagrams showing (A) a prototypical GFP-replicon, (B) an MVA-P1 construct, and (C) a method of replicon encapsidation.

FIG. 2. Replicon preparations contain no replication-competent wild-type polio virus.

FIG. 3. In vivo infection of human D54-MG tumor cells by encapsidated replicons. Each bar represents the IL-6 levels from an individual mouse.

FIG. 4. (A) GFP fluorescence of human D54-MG malignant glioma cells infected by GFP replicons in vitro. White arrows indicate cells undergoing vacuolization (B) Hoechst stain of human malignant glioma cells infected in vitro. The blue fluorescence characteristic of the Hoechst stain was adjusted to purple for better contrast. White arrows indicate condensed, brightly staining nuclei. (C) Combined image of GFP green fluorescence and Hoechst DNA staining. The white arrows show the association of the condensed nuclei highlighted in Panel C with green fluorescence in Panel D.

FIG. 5. In vivo growth inhibition of D54-MG tumors by replicons.

FIG. 6. Survival of mice implanted with glioma cells treated with replicons ex vivo prior to transplantation.

FIG. 7. Survival of mice implanted with glioma cells and subsequently treated in vivo with replicons.

FIG. 8. Histology of replicon-treated D54-MG tumor cells.

FIG. 9. Histology of metastasized, replicon-treated D54-MG tumor cells.

FIG. 10. Dose-dependent inhibition of replicon infection by exposure to the anti-CD155 monoclonal antibody D171.

### DETAILED DESCRIPTION OF THE INVENTION

The present invention relates to compositions comprising poliovirus-based replicons with or without a transgene and their use in lysing target tumor cells. The invention further provides for the use of such replicons in cancer therapy, wherein tumors or tumor cells are contacted with replicons and such replicons induce lysis of the tumor cells.

A. Composition

According to the present invention, the term oncolysis and its grammatical equivalents describe the death of one or more tumor cells by apoptotic, non-apoptotic or other mechanisms.

According to the invention, replicons are poliovirus-based polynucleotides which possess oncolytic activity towards a variety of different tumor cells. Replicons may be a naked nucleic acid or fully encapsidated. Replicons of the invention lack a wild type poliovirus nucleic acid necessary for encapsidation of the virus. Consequently, newly encapsidated replicons cannot be produced following initial cell entry in the absence of the missing RNA. Replicons may lack this nucleic acid as a result of any modification of the wildtype poliovirus nucleic acid including, but not limited to, deletions, insertions, and substitutions. The lacking nucleic acid may be as small as a single nucleotide. A non-limiting example of a replicon lacking a nucleic acid this small is one in which a point mutation renders an encoded capsid protein insufficient or ineffective for encapsidation. Replicons of the invention may comprise a substantially deoxyribonucleic acid (DNA) or substantially ribonucleic acid (RNA) genome.

In prefered embodiments of the invention, replicons lack a wild type poliovirus nucleic acid that encodes at least a portion of a protein that is required for encapsidation. The absence of this nucleic acid may block translation of the required protein. Alternatively, the absence of this nucleic acid may result in expression of a nonfunctional form of the required protein. According to the invention, a “portion of a protein” may be as small as a single amino acid. Thus, the smallest nucleic acid that can be lacking is a single nucleotide. For example, the invention contemplates a base substitution at a single position such that the sequence of the resulting polynucleotide encodes a capsid protein that differs in one amino acid from it's wildtype counterpart and is incapable of encapsidating a replicon. In this context, the missing nucleic acid is a single nucleotide that comprises a codon for an amino acid that is critical to capsid protein function.

Proteins necessary for replicon encapsidation include proteins that are part of the capsid structure. Examples of such proteins are those encoded by the VP1, VP2, VP3, and VP4 genes of the poliovirus P1 capsid precursor region, the Vpg protein, and those proteins that are necessary for proper processing of structural proteins of the capsid structure, such as the proteases responsible for cleaving the viral polyprotein.

Replicons of the invention are typically introduced into a cell in an RNA form. Encapsidated replicons are able to enter cells via interaction of the capsid proteins with poliovirus receptor, e.g. the hPVR protein (CD155). Replicons of the invention are fully capable of RNA replication (amplification) upon introduction into cells and translation, in the correct reading frame, of the single polyprotein through which expression of the entire replicon genome occurs. Translation of replicon sequences may be transient, usually lasting only about 24-48 hours. High levels of replicon-encoded proteins can accumulate during the translation period.

Replicons of the invention that lack most or all of the capsid gene sequences but do not contain substituted non-poliovirus sequences possess a cell autonomous oncolytic activity, i.e. non-infected cells nearby replicon-infected tumor cells are not killed. The oncolysis of replicon-infected cells is a cell autonomous event having substantially no bystander effect. Without being restricted to any particular mechanism of action, the oncolysis of replicon-infected cells may result wholly from intracellular poliovirus genetic material (i) presence, (ii) amplification, (iii) translation or (iv) combinations thereof.

Replicons of the invention may additionally comprise a heterologous nucleic acid with a minimum length of one nucleotide. According to the invention, a heterologous nucleic acid is any nucleic acid that is not present in the genome of wild-type poliovirus. Thus, the invention contemplates a replicon having a transgene, a site-specific mutation (e.g. deletion, insertion, or substitution), a restriction site, a site-specific recombination site (e.g. loxP, FRT, and RS), an expression control sequence, or combinations thereof. For example, a replicon may be prepared having a transgene and the restrictions sites necessary for its integration. Alternatively, a replicon may lack heterologous sequences. This terminology relates principally to replicons that lack a sequence required for encapsidation and lack nucleic acids of exogenous origin. For example, this encompasses replicons from which the P1 gene has been deleted even though the sequence at the splice junction is not a wild-type sequence per se.

The invention contemplates the use of a wide variety of transgenes. In accordance with the instant invention, a transgene is a nucleic acid, the sequence of which is not present in the wild type poliovirus genome. Transgenes may confer or enhance oncolytic activity by various means. Thus, a transgene for use in the invention may encode a cytotoxic protein which may be directly toxic to cells in which it is expressed, such as urokinase, tumor neucrosis factor-α (TNF-α) or interleukin-4 (IL-4). Thus, a cytotoxic protein according to the invention does not require exogenous substrates to promote cell death. A transgene of the invention may also encode a protein which itself is non-toxic, but can convert a prodrug to a toxic product (a “prodrug converting protein”), such as herpesvirus thymidine kinase (HSV-TK), purine nucleoside phosphorylase or cytosine deaminase. A transgene for use in the invention may also encode a product that enhances the oncolytic activity of the poliovirus by mechanisms now known or later discovered. These examples are not limiting and additional embodiments in which oncolysis of infected or non-infected cells is produced or enhanced by non-poliovirus sequences (i.e. transgenes) may exist. In preferred embodiments, non-poliovirus sequences are substituted for the capsid (P1) gene in the poliovirus genome.

The invention further contemplates the use of transgenes for other purposes. Thus, a transgene of the invention may also encode markers such as luciferase, an autofluorescent protein (e.g. green fluorescence protein), and β-glucuronidase. A transgene for use in the invention may also encode an immunogen. Nonlimiting examples of immunogens include hepatitis B surface antigen, influenza virus hemaglutinin and neuraminidase, human immunodeficiency viral proteins, respiratory syncycial virus G protein, bacterial antigens, chimeric foreign genes and B and T cell epitopes. Nonlimiting examples of human immunodeficiency viral proteins include gag, pol, and env. Nonlimiting examples of bacterial antigens include tetanus toxin, diphtheria toxin, cholera toxin, mycobacterium tuberculosis protein B antigen and fragments thereof. In some embodiments of the invention, the transgene encodes an antigen from an infectious agent.

Replicon infection produces various cytopathic effects in cultured and primary isolates of human glioma cells (those obtained directly from patients). These effects include cell rounding, reduced translation of host cell genes, membrane perturbations, increased vacuolization, and ultimately cell death, usually within about 24 hours. The literature contains conflicting reports regarding whether the cytopathic effects and cell death associated with wild-type poliovirus infection are associated with apoptosis or are caused by another virus-specific mechanism that does not display all the hallmarks of an apoptotic pathway. Similarly, the mechanism of replicon cytopathicity is not completely understood. Without being restricted to any particular mechanism of action, the oncolytic effect of replicons is likely to be dependent on both the presence of hPVR on the cell surface to permit cell entry and an appropriate intracellular environment to allow nucleic acid amplification. Replicons retain viral genes that take over the host cell protein translation machinery. It has been proposed that inhibition of host cell protein synthesis triggers apoptosis (Goldstaub D et al., 2000, Mol. Cell Biol. 20(4):1271-1277). However, without being restricted to any particular mechanism of action, apoptotic cells were not detected in histological sections of D54-MG tumor cells from the brains of scid mice that had been injected with replicons having a transgene encoding green fluorescent protein (GFP), despite the use of a sensitive TUNEL assay (see below). Therefore, the present invention encompasses replicons that exert an oncolytic effect through an apoptotic, non-apoptotic or other mechanism.

In some preferred embodiments of the invention, replicons comprise RNA and are encapsidated. Preferably, replicon vectors have a deletion of the capsid (P1) gene and are derived from the RNA genome of poliovirus type 1, type 2, type 3 or combinations thereof. Further, non-poliovirus sequences may be substituted for part or all of the capsid (P1) gene such that the portion of the capsid (P1) gene which remains, if any, is insufficient to support encapsidation in vivo. The capsid (P1) gene may be replaced by a non-poliovirus nucleic acid molecule (transgene) encoding a protein of interest. Non-limiting examples of such transgenes include genes encoding markers, such as luciferase, green fluorescence protein, and β-glucuronidase; enzymes such as HSV-TK and purine nucleoside phosphorylase; biologically active molecules such as TNF-α, IL-4, IL-6, and granulocyte/macrophage colony-stimulating factor (GM-CSF); protein or non-protein-based inducers of HPVR accumulation in target cells; and protein or non-protein-based inducers of intracellular factors that enhance or are required for replication of the replicon RNA genome.

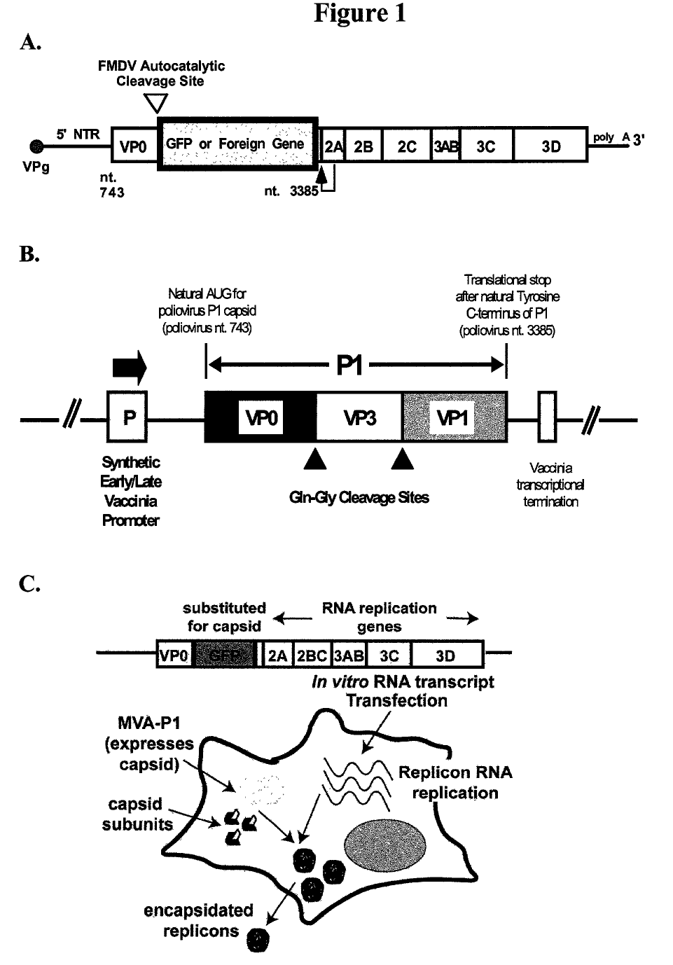

A non-limiting example of a prototype replicon is shown in FIG. 1A. According to this example, the replicon genome is derived from the poliovirus type 1 Mahoney RNA genome. The replicon RNA retains the features of wild-type poliovirus required for replication of the RNA genome and translation of proteins. At the 5′ end, a small peptide known as VPg is covalently linked to the RNA genome; no methyl guanosine cap exists at the 5′ end of the RNA molecule. A 742 nucleotide nontranslated region of RNA sequence is positioned upstream of the single, long open reading frame. The nontranslated region contains the internal ribosome entry site (or IRES), which mediates cap-independent translation of the replicon proteins in the host cell. Most of the capsid P1 gene of the wild-type virus is substituted with sequences encoding a foreign gene of interest, such as the GFP marker gene. The foreign gene is positioned between the 3′ end of the VP0 gene (one of the individual capsid genes; VP3 and VP1 are deleted from the repli con construct) and the 5′ end of the poliovirus 2A gene. The viral proteins, as well as the foreign protein of interest, are translated as part of a long polyprotein molecule. The polyprotein is subsequently cleaved by proteases encoded by the replicon RNA to generate the individual foreign protein and other viral proteins (2A, 2B, 2C, 3AB, 3C, 3D) required for amplification of the RNA. In the non-limiting example construct shown, the GFP protein is liberated from the polyprotein through an autocatalytic cleavage at the N-terminus (mediated by a short engineered peptide derived from Foot and Mouth DiseaseVirus (FMDV), or that has autocatalytic proteolytic activity) and a second intramolecular cleavage at the C-terminus, which is mediated in cis by the 2A protease. Because the replicon RNA genomes lack the coding sequence for a full-length, functional capsid protein, replicons cannot self-propagate and spread from cell to cell. Encapsidation of the replicon RNA genomes requires intracellular expression of the capsid in trans from a separate vector.

Encapsidated replicons may be produced by introducing both a replicon nucleic acid and a complementing virus vector that provides missing sequences necessary for encapsidation in trans to a host cell. Use of this complementing virus allows for generation of large scale, high titer stocks of encapsidated replicons. Methods which may be used to prepare encapsidated replicons have been described in inter alia Porter et al., 1993, J. Virol. 67:3712-3719; Porter et al., 1995, J. Virol. 69:1548-1555; WO 96/25173; U.S. Pat. No. 5,614,413; U.S. Pat. No. 5,817,512; U.S. Pat. No. 6,614,413; all of which are incorporated herein by reference. Encapsidated replicons may be produced in suitable host cells, for example, by using a modified vaccinia virus (MVA) that encodes a poliovirus type 1 Mahoney capsid precursor protein (MVA-P1)(FIG. 1B), a Sabin capsid precursor protein or an engineered capsid.

An example of a recombinant MVA is shown in FIG. 1B. Recombinant Modified Vaccinia Ankara (MVA) which expresses the poliovirus type 1 Mahoney cap sid (P1) contains the cDNA encoding P1 under the control of a synthetic early/late Vaccinia virus promoter (Carroll and Moss, Biotechniques 19:352). The inserted gene is followed on the 3′ end by transcriptional termination signals for Vaccinia virus. The entire construct is flanked by sequences homologous to the deletion site III region of MVA, which direct homologous recombination of the recombinant gene into the MVA genome (Sutter and Moss, Proc. Natl. Acad. Sci. USA 89:10847). The recombinant P1 gene spans the natural length of the poliovirus type 1 Mahoney capsid coding sequences, from nucleotide 743 to 3385. A synthetic translational stop codon has been inserted immediately downstream of the codon for the tyrosine amino acid that is the natural C-terminus of P1. Upon translation in the host cell, the P1 capsid polyprotein is cleaved at glutamine-glycine amino acid pairs to generate the individual capsid proteins VP0, VP3, and VP1 which assemble into a capsid shell. The proteolytic cleavage event is dependent upon the viral protease 3CD. For production of encapsidated replicons, the 3CD protease is expressed from the replicon RNA genome.

In some embodiments of the invention, replicons are derived from a poliovirus vector comprising site-specific recombination sites and a nucleic acid that is necessary for encapsidation, e.g. the P1 gene, wherein the recombination sites flank said nucleic acid. A replicon is produced by contacting the poliovirus with a site-specific recombinase that is capable of excising the nucleic acid that is necessary for encapsidation. The invention contemplates accumulation of sufficient capsid protein prior to the recombination event to permit encapsidation of the recombined genome. To reduce or prevent leak-through encapsidation of replication-competent poliovirus vector, the length of the nucleic acid between the recombination sites may be adjusted such that the unrecombined genome is too large to be encapsidated.

The present invention contemplates the use of other capsids for encapsidation. Non-limiting examples include capsid proteins sharing 90% amino acid similarity to wild type poliovirus capsid and capsid proteins from the picomavirus family. In addition, the invention contemplates use of capsids conjugated with antibodies or other cell surface protein-binding molecules that may allow targeting to specific cells of interest.

Although many human tumor lines and tumor explants tested are susceptible to infection with replicons, variation in susceptibility exists between tumor types. The variation may be related to the amount of receptor present on the cell surface. Thus, target cell susceptibility to replicon infection may be limited by insufficient expression of hPVR or other reasons. To overcome such potential limits and to permit replicon entry into a broader or different range of target cells, the invention further provides means of masking and/or modifying the surface of a replicon to permit entry into a broader or different range of cells. In some embodiments of the invention, the capsid provided by complementation may be modified. For example, a capsid with an integrin-binding domain on an exposed surface loop of the capsid may be used. In some embodiments of the invention, replicons may be delivered to target cells via lipid vehicles, polylysine vehicles (Kollen et al., 1996, Hum. Gene. Ther. 7:1577-1586), synthetic polyamino polymer vehicles (Goldman et al., 1997, Nat. Biotechnol. 15:462-466), and molecular conjugates (Roux P et al., 1989, Proc. Natl. Acad. Sci. USA 86 (23):9079-9083). For example, liposomes or polyethylenimine (PEI) may be used to deliver replicons to targeted cells, e.g. tumor cells.

In some embodiments of the invention, encapsidated or unencapsidated replicons may be delivered to target cells via delivery vehicles comprising cationic amphiphiles such as lipids, synthetic polyamino polymers (Goldman et al., 1997, Nat. Biotechnol. 15:462-466), polylysine (Kollen et al., 1996, Hum. Gene. Ther. 7:1577-1586) or molecular conjugates such as a biotinylated anti-major histocompatibility complex (MHC)(Roux P et al., 1989, Proc. Natl. Acad. Sci. USA 86 (23):9079-9083).

In some embodiements of the invention, the delivery vehicle comprises a bifunctional complex for linking the delivery vehicle to a target cell (see e.g. O'Riordan et al., Aug. 12, 1999, WO 9940214). A bifunctional complex comprises an element that is capable of binding a replicon, a linker, and an element that is capable of binding a cell surface molecule displayed on the surface of the target cell. Non-limiting examples of replicon binding elements include poliovirus receptor and antibodies raised against a poliovirus capsid protein. Linkers may comprise a chemical linker that can attach to the other elements via covalent and/or ionic linkages. Examples of covalent linkers include, but are not limited to, sulfhydryl and maleimide linkages. Examples of ionic bond linkages include, but are not limited to, cationic molecules such as poly-L-lysine (PLL) and polyethylene glycol-PLL (PEG-PLL). Additional linkers include biocompatible polymers having an average weight of 200 to 20,000 daltons which may be chemically modified to be used as linkers (O'Riordan et al., Aug. 12, 1999, WO 9940214). Non-limiting examples of target cell surface moieties to which the target cell surface binding element may be directed include the folate receptor (Melani et al., 1998, Cancer Res. 58(18):4146-4154), the transferrin receptor (Debinski and Pastan, 1992, Cancer Res. 52:5379-5385), the fibroblast growth factor (FGF) receptor (Goldman et al., 1997, Cancer Res. 57:1447-1451), the epidermal growth factor (EGF) receptor (Bell et al., 1986, Nucleic Acids Res. 21:8427-8447), the c-kit receptor (Schwarzenberger et al., 1996, Blood 87:472-478), the erythrocyte growth factor receptor (Shimizu et al., 1996, Cancer Gene Therapy 3:113-120), the polymeric Ig receptor (Piskurich J F et al., 1995, J. Immunol 154(4):1735-1747), the erythropoietin receptor (Yoshimura and Misawa, 1998, Curr Opin Hematol. 5:171-176), a purinoceptor (O'Reilly, 1998, Br. J. Pharmacol. 124:1597-1606), and various enzymes (e.g. metalloproteases).

Prior to use in animals or humans, replicon infectivity and oncolytic activity are tested in vitro, ex vivo, in vivo or combinations thereof. All assays include use of tumor cells that are as similar as possible to the actual types of tumor cells in the individual to receive replicon therapy. In vitro testing comprises infectivity and/or oncolytic activity assays using cultured cells. Ex vivo testing comprises infectivity and/or oncolytic activity assays using primary tumor cells, obtained from biopsy material from tumors in individuals. Biopsy-derived cells may or may not be passaged prior to the ex vivo assay.

Replicon infectivity and oncolytic activity may be tested in vivo by using human tumor lines xenografted into immunocompromised mice (scid or nude), which do not reject the tumors. Transplanted cells may be introduced into the host as single cells, clusters, or tumor explants. The use of human tumor lines ensures that the tumors will be susceptible to replicon infection. Replicon effects in animals can also be tested by using transgenic mice which express the human poliovirus receptor. These animals develop a paralytic disease that mimics poliomyelitis in humans when they are infected with poliovirus. Therefore, they are a suitable model animal for studying toxicity and/or disease-causing capacity of replicons in vivo. However, because these animals are immunocompetent, they will not accept human tumor xenografts. Therefore, any cancer cells introduced to these animals must be derived from mice and must be modified so that they express the human poliovirus receptor if direct infection of those cells is desired.

In addition to infectivity and oncolytic activity, replicon titer and dosing will be determined prior to use in treatment. Replicons may be titered by (i) providing replicons of unknown titer encoding a marker, (ii) providing replicons of known titer encoding the same marker, (iii) exposing dilutions of each to cells, and (iv) comparing the expression level of the marker in cells exposed to dilutions of replicons of unknown titer with the expression level of the marker in cells exposed to dilutions of replicons of known titer. The marker may be encoded by a poliovirus gene such as 3CD or a transgene such as GFP. Detection of marker expression may be achieved by any commonly known and available technique such as immunoassays, enzymatic assays or fluorescence detection.

Oncolytic acitivity and/or replicon titer may be ascertained using limited dilution/cytopathic effect assays. In such assays, a known quantity of cells are infected with a preparation of wild type poliovirus or replicons of known oncolytic activity and known titer. In parallel, a known quantity of cells is infected with replicons of unknown oncolytic activity/titer (“test replicons”). The cytopathic effects of the two may be compared to semi-quantitatively determine the oncolytic activity and/or titer of the test replicons.

Dosing may be determined by extrapolation from infectivity and oncolytic activity data generated in mice, as well as from toxicity data generated in the transgenic mice and primates.

Various combinations of the preceding infectivity and oncolytic activity assays are contemplated.

An important feature of replicons is their ability to effectively distribute within the brain and CNS (Bledsoe A W et al., 2000, J. Neuro Virol. 6:95-105; Bledsoe A W et al., 2000, Nature Biotechnology 18:964-969). The extension of survival following administration of replicons to animals with intracranial tumors was undoubtedly due to the inherent capacity of replicons to effectively infect both at the site of implantation as well as sites in which the tumor cells had begun to metastasize. The physical properties of the replicons, small virus particles (30 nM) which do not contain an envelope, may facilitate distribution in tissues from the site of administration. Poliovirus has the inherent capacity to cross the blood-brain barrier to gain entry into the brain and CNS (Yang W X et al., 1997, Virol. 229:421-428). Replicons given intraspinally can access most compartments of the CNS including infection of the cells in the lower brain stem Collectively, replicons based on poliovirus take advantage of the evolution of this virus to successfully move to tissues outside and within the CNS.

B. Method

The present invention further relates to methods of using poliovirus-based replicons to kill and/or inhibit the growth of tumor cells comprising contacting the target cells with (i) replicons and (ii) optionally one or more factors which increase replicon infectivity of the target cells.

Although applications of the invention toward oncolysis of specific tumors are described in the examples, the invention contemplates the broad use of poliovirus-based replicons to kill and/or inhibit the growth of a variety of tumor cells. According to the invention, “tumor cells” means neoplastic or benign growths of human cells wherein neoplasia refers to malignant growths. Thus, “tumor” encompasses both neoplastic and benign growths. Non-limiting examples of tumors which may affected (e.g. growth inhibited or killed) by replicons are those derived from cells of the circulatory system, reproductive system, nervous system, gastrointestinal system, respiratory system, endocrine system, immune system, bone, skin, liver, breast, ovary, testes, prostate, head, mouth, brain, and spinal cord and pancreas. Non-limiting examples of tumors which may affected (e.g. tumor cells are growth inhibited and/or killed) by replicons are cervical adenocarcinomas, osteosarcomas, malignant gliomas, astrocytomas, oligodendogliomas, ependymomas, breast carcinomas, melanoms, gliosarcoma, meningioma, melanomas, and squamous cell carcinomas.

The present invention contemplates a wide range of delivery methods by which tumor cells may be infected with replicons. Encapsidated replicons may be administered inter alia surgically, intracranially, intraspinally, intramuscularly, intratumorally, orally, nasally, rectally, vaginally, topically, intravenously, by injection or by inhalation.

Preferably replicons comprise RNA, possess an inherent capacity to kill tumor cells in vivo, and may limit the growth of implanted tumor cells in vivo. More preferably, replicons are encapsidated prior to administration. Replicons are capable of infecting tumor cells, initiating an RNA replication cycle, producing replicon-encoded proteins, and triggering oncolysis.

In one embodiment of the invention, replicons may be used to inhibit growth of and/or kill malignant gliomas in human patients. Accordingly, replicons are capable of infecting malignant gliomas, initiating an RNA replication cycle, producing replicon-encoded proteins, and triggering oncolysis of the infected cells. Replicons may be introduced directly into the brain of glioma patients to selectively infect cells associated with the malignancy without risk of deleterious effects on other cells of the brain. The safety of this approach has been demonstrated by Bledsoe et al. in an animal model system where replicons were directly delivered to the CNS of transgenic mice susceptible to poliovirus infection (Bledsoe et al., 2000, J. Neurovirol. 6:95-105). In this study replicons were administered at substantially higher doses than those needed to cause a paralytic poliomyelitis infection in the animals, without development of disease or pathological symptoms of infection (Bledsoe et al., 2000, J. Neurovirol. 6:95-105).

A major determinant of susceptibility of cancer cells to encapsidated replicon infection is the presence of the human poliovirus receptor (hPVR, also known as CD 155) on the surface of the cells (Mendelsohn C L et al., 1989, Cell 56:855-865; Ren R et al., 1990, Cell 63:353-362). The human poliovirus receptor has been well characterized, and its expression on the surface of cells has been shown to be required for infection by wild-type poliovirus (Mendelsohn C L et al., 1989, Cell 56:855-865). According to one embodiment of the invention, an interaction between replicons and CD 155 must occur in order for the cancer cells to become infected and express proteins encoded by the RNA genome.

The invention also provides that one or more agents that enhance replicon infectivity or oncolytic activity may be administered to a subject. Preferably, this administration occurs prior to or concurrent with replicon administration. Non-limiting examples of such agents include hemin and retinoic acid. The instant inventors have observed that pre-treatment of cancer cell lines with retinoic acid results in enhancement in the number of cells that become infected with the replicons. In the case of hemin, it has been shown that K562-Mu erythroleukemia cells, which are normally resistant to poliovirus-mediated cytopathic effects, become susceptible to virus-induced cell lysis after growth in hemin (Benton et al., 1996, J. Virol. 70:5525-5532). Another approach is to pretreat the tumor in vivo with a vector comprising a sequence that encodes hPVR where the vector targets the tumor cells. Tumor cells expressing hPVR from the exogenously supplied gene may display more receptor on the cell surface.

In some embodiments of the invention, the individual is treated with one or more additional vectors that enhance replicon infectivity, oncolytic activity or both. Preferably, such vectors are administered prior to or concurrent with replicon administration. In one non-limiting example, a vector that leads to increased production of hPVR in tumor cells may be used. Such a vector may increase the amount of hPVR displayed on the surface of tumor cells, thereby enhancing their susceptibility to replicon infection. Such a vector alternatively may induce or increase the production of intracellular proteins required for replication of the replicon RNA genome. According to another non-limiting example, a vector may be used which leads to the production of a cytotoxic agent in tumor cells. Production of such a cytotoxic agent may or may not depend on the presence of replicons or replicon-encoded gene products. In addition, production of such a cytotoxic agent may or may not occur in the presence or absence of replicons.

Replicon infectivity and oncolytic activity are tested prior to use in individuals by one or more of the methods described above.

C. Advantages

The replicon system offers several advantages among candidate gene therapy and viral-mediated strategies for cancer treatment. First, RNA replicons cannot act as mutagens since they cannot integrate into or recombine with human chromosomal DNA. DNA vectors such as HSV-1 and Adeno-associated virus, on the other hand, may integrate into or recombine with chromosomes, potentially inactivating essential genes or activating latent genes and even resulting in cancer development.

Second, RNA replicons are not capable of spreading in the host beyond the initially infected cell. This contrasts with a recently described study which proposes the use of replication-competent polioviruses containing an internal ribosome entry site (IRES) element from the related human rhinovirus substituted in place of the corresponding poliovirus element as a therapy for malignant gliomas in humans (Gromeier, M et al., 2000, Proc. Natl. Acad. Sci. USA 97(12):6803-6808). In that case, the resultant viruses are capable of replication and cell-to-cell spread, although the hybrid viruses appear to be incapable of causing neuropathogenesis. Replicons of the invention differ because newly encapsidated replicons cannot be produced following cell entry absent the missing sequence necessary for encapsidation. Therefore, there is no concern of replicon spread away from the site of introduction in an uncontrolled manner. In addition, replicons cannot “evolve” in vivo during multiple rounds of replication and infection, thereby regaining the capacity for causing neuropathogenesis.

Third, although replicons are not capable of spread, they do retain all of the viral genes required for amplification of the RNA genome and production of high levels of vector-encoded proteins. These proteins are produced during a transient burst of vector gene expression lasting only 24-48 hours. This allows a brief period of high level protein expression, but avoids potential detrimental effects which may arise due to sustained expression for longer periods of time. These features contrast with other viral vector systems which may express proteins for a longer period and/or which may persist in a latent state for very long periods. A short, predictable period of expression also enables the use of replicons encoding other genes, such as suicide genes, that, alone or in combination with prodrugs, could lead to an amplified anti-tumor effect without side effects which may arise during sustained expression of such a gene. The combination of short term gene expression coupled with the inability for spread from cell to cell supports the contention that replicons are safe.

Fourth, replicons pose no greater pathological threat than the current vaccination practice using attenuated poliovirus. Similarly, oncolysis of tumor cells by replicons is expected to have no more severe side effects than current vaccination practices using attenuated poliovirus. Safe use of replicons has been demonstrated when administered in the periphery and after intracranial or intraspinal inoculation (Bledsoe A W et al., 2000, J. Neuro Virol. 6:95-105; Bledsoe A W et al., 2000, Nature Biotechnology 18:964-969; Jackson C A et al., 2001, “Repetitive intrathecal injections of poliovirus replicons result in gene expression in neurons of the central nervous system without pathogenesis”, Human Gene Therapy 12:1827-1841).

### EXAMPLES

### Example 1

### The Cytopathic Effects of Replicons on HeLa-H1 Cells

Encapsidated replicons were prepared by previously described methods (FIG. 1C)(Porter et al., 1993, J. Virol. 67:3712-3719; WO 96/25173; U.S. Pat. No. 5,614,413; U.S. Pat. No. 5,817,512; U.S. Pat. No. 6,614,413, incorporated herein by reference). Replicons were serially passaged on HeLa-H1 cells in the presence of vaccinia virus P1 (VV-P1).

Alternatively, replicons were prepared by the previously described methods except that a recombinant modified vaccinia Ankara that expresses the poliovirus type 1 capsid precursor protein (MVA-P1) was used to supply the capsid. Briefly, replicon RNA run-off transcripts were generated in vitro from cDNA templates by using bacteriophage T7 RNA Polymerase. The RNA transcripts were transfected (using DEAE-Dextran) into HeLa-H1 cells that had been infected previously for 2 hours with MVA-P1. After an overnight incubation at 37° C., a freeze-thaw lysate of the infected cells was generated, and encapsidated replicons were recovered in the media supernatant following a clarification spin in a microcentrifuge at 12,000× g. The recovered replicons were used directly for experiments or to infect new monolayers of HeLa-H1 cells that had been infected with MVA-P1 to generate larger stocks through serial passage.

Prior to injection in animals, encapsidated replicons were filtered through a 0.2 micron filter (Nalgene, Rochester, N.Y.) and treated with detergent (1% Triton X-100) to inactivate any recombinant MVA-P1 in the preparations. The replicon preparation was concentrated by ultracentrifugation at 55,000 rpm in a SW-55 rotor (Beckman Coulter, Inc., Fullerton, Calif.) through a 30% sucrose cushion to concentrate encapsidated replicons as described previously (Proter D C et al., 1995). Replicon preparations were resuspended in phosphate buffered saline (PBS), pH 7.2, and were stored at −70° C. prior to use.

Replicons were titered by two methods. According to the first method, replicons were titered using an immunoprecipitation assay in which the expression level of poliovirus 3CD protein in HeLa-H1 cells infected with poliovirus was compared with the 3CD expression level in HeLa-H1 cells infected with various dilutions of replicons. A standard curve of 3CD expression was first determined from the known amounts of poliovirus using phosphorimagery of immunoprecipitated protein gel bands. Next, phosphorimagery intensity data from the replicon immunoprecipitations was located on the standard curve which was then translated back to a titer.

Replicons were also titered by plating dilutions of the replicons on a known number of HeLa-H1 cells. The most diluted sample which still kills all of the cells in a known number of HeLa-H1 cells was then be used to generate a titer for the replicon preparation based on this cell killing activity (reported as “infectious units”).

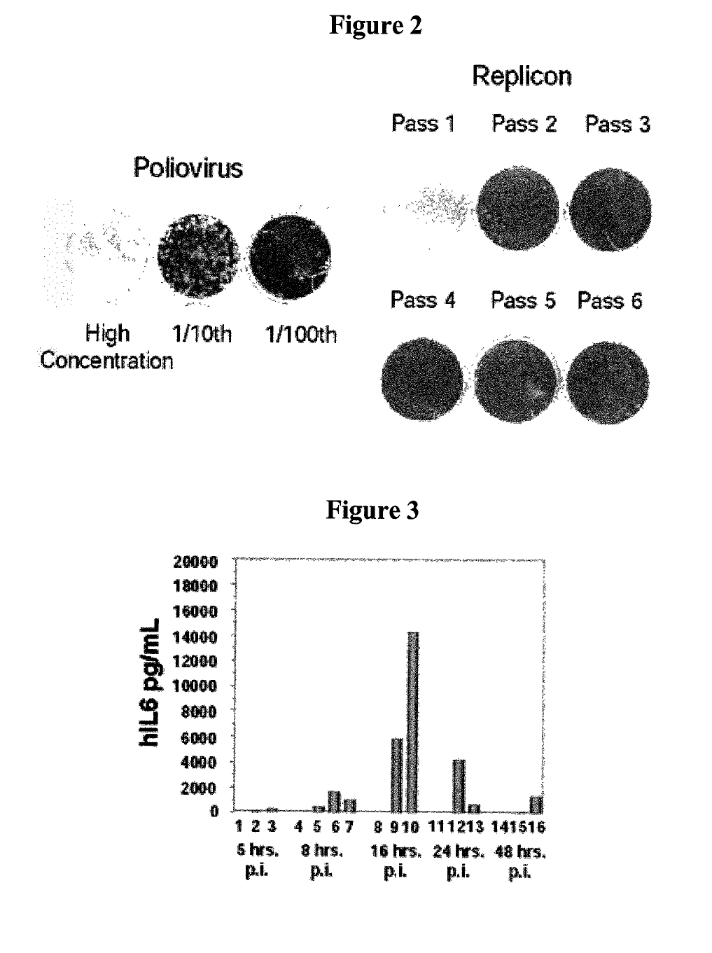

Using a replicon stock of known titer, HeLa cells were infected with the encapsidated replicons at a multiple of infection (MOI) of 2. Infected cells demonstrated a clear cytopathic effect by rounding beginning at approximately 6-8 hours post infection. At 24 hours post infection all of the cells were detached from the tissue culture plate. Subsequent passage of the supernatant after the first round on HeLa cells resulted in no further cytopathic effect on the cells (FIG. 2). In this assay, if replicon preparations contained poliovirus, completely white wells or “spots” would appear at times later than Pass 1. The absence of such spots is indicative of the lack of wild type or otherwise replication-competent poliovirus (FIG. 2). FIG. 2 shows typical cytopathic effects associated with wild-type poliovirus infection. Plating of wild-type poliovirus at low multiplicities of infection results in the development of plaques when cells are grown under an agarose overlay. Plaque formation is diagnostic for cell-to-cell spread. Replicons, however, do not form plaques since they are limited to a single round of infection and cannot spread to neighboring cells.

### Example 2

### In vitro Infection of Human Cancer Cell Lines with Replicons

Wild type poliovirus is capable of infecting a wide variety of human tumor cells (Gromeier M et al., 2000, Proc. Natl. Acad. Sci. USA. 97(12):6803-6808; Solecki D et al., 1999, J Biol. Chem. 274(3)1791-1800). Since replicons are encapsidated and maintain an RNA amplification phenotype (but not the ability to form new capsids), experiments were performed to determine whether replicons have the same or a similar range of infectivity as intact poliovirus. Replicons derived from type 1 poliovirus comprising a transgene encoding firefly luciferase were administered to established tissue culture lines of human gliomas (D24 and U251) and primary tumor cells that had been resected from human patients. The primary tumor cells were analyzed after 3 passages or less in tissue culture. In this assay, detection of luciferase activity in infected cells is dependent upon infection (Porter D C et al., 1998, Virology 243(l):1-11). Both the established cell lines and the primary cultures of malignant gliomas could be infected, as measured by detection of abundant luciferase activity from the cell cultures (Table 1). Established glioma cell lines tested included D54MG cells and U251 cells, which had been maintained in tissue culture for many passages. Most interestingly, primary tumors from patients, including two different astrocytomas, as well as an ependymoma, were also readily infected. The astrocytoma 4/99 was a tumor that was assayed by infection following immediate removal from a patient with no interval for in vitro culture. The other primary tumors tested had been in tissue culture for two to three passages. Visual inspection of all of the cultures following infection with replicons revealed a pronounced cytopathic effect that resulted in death after approximately 24 hours of infection, similar to that seen for the HeLa-H1 cells (FIG. 2).

TABLE 1

| Cell Line/Type | Control Replicon | Luciferase Replicon |
|---|---|---|
| Glioma Lines: | | |
| D54-MG | background | 3,932,000 light units |
| U251 | background | 525,720 light units |
| Primary Tumors: | | |
| Astrocytoma¹ | background | 699,254 light units |
| Ependymoma¹ | background | 1,199,792 light units |
| Astrocytoma (4/99)² | background | 203,480 light units |

¹Tumor cells were passaged 2-3 times in culture to induce poliovirus receptor expression.
²Tumor cells were not passaged in culture to induce poliovirus receptor expression.

### Example 3

### In vitro Infection of Human Cancer Cell Lines with Replicons

A more extensive demonstration of the variety of cell lines which can be infected by replicons is provideed in Table 2. The capsid (P1) gene of poliovirus in these replicons was substituted with a nucleic acid encoding GFP, luciferase or hIL-6. The resulting replicons were encapsidated and tested for their ability to infect, express the non-poliovirus gene, and kill infected cells.

For analysis of in vitro infection of human tissue culture cell lines, tumor cells were plated in 6-well tissue culture dishes in DMEM or DMEM/F 12 as appropriate for the particular cell line. For infection, encapsidated replicons were adsorbed to the cell monolayers in 0.8 mL of medium for 1 hour, and then volumes were increased to 2 mL for further incubation at 37° C. Tumor cell lines were infected with 10 infectious units per cell as determined by titer assay on HeLa-H1 cells. Incubations were allowed to proceed for 24-48 hours, and the monolayers were observed for relative cytopathic effects and cell killing as determined by cell rounding and detachment from tissue culture dishes. The percentage of cells killed was noted for each cell line in comparison to uninfected controls. For patient tumor cell samples, infections were performed in a similar manner, except that the multiplicity of infection was not determined because of the characteristics of the primary cells, which often grew in scattered clumps. Because of the variation in growth of the primary lines in vitro and the variation in multiplicities of infection used (5-100 i.u./cell), the determination of a percentage of cells killed was not possible. We did note that in each case, however, replicon infection caused death of greater than 25% of the cells in the culture after 48 hours.

TABLE 2
In Vitro Killing Human Tumor Cells of
Diverse Origin with Encapsidated Replicons

| Cell Line | Tumor Type | Cell Killing^{a} |
|---|---|---|
| CNS Tumor Cell Lines: | | |
| D54-MG | Malignant glioma | ***** |
| U251-MG | Malignant glioma | ***** |
| U373-MG | Astrocytoma | ***** |
| D32GS | Gliosarcoma | **** |
| SK-N-MC | Neuroblastoma | ***** |
| CH-157-MN | Meningioma | **** |
| U118-MG | Glioblastoma multiforme | **** |
| IMR-32 | Neuroblastoma | ** |
| Hs-683 | Anaplastic glioma | * |
| Patient Tumors^{b}: | | |
| UAB8129 | Glioblastoma multiforme | Y |
| UAB1016 | Oligodendoglioma | Y |
| UAB9756 | Glioblastoma multiforme | Y |
| TCH0353 | Pilocytic astrocytoma | Y |
| TCH5905 | Ependymoma | Y |
| 99040123^{c} | Anaplastic astrocytoma | Y |
| 01011015^{c} | Glioblastoma multiforme | Y |
| 010301016^{c} | Glioblastoma multiforme | Y |
| 010201010^{c} | Meningioma | Y |
| Non-CNS Tumor Cell Lines: | | |
| SK-MEL-2 | Melanoma | **** |
| SK-MEL-21 | Melanoma | *** |
| SK-MEL-28 | Melanoma | ** |
| BT20 | Breast | **** |
| HT1080 | Fibrosarcoma | *** |
| DLD-1 | Colon | *** |
| SQ-20-B | Squamous cell carcinoma | *** |
| SK-Hep1 | Lung adenocarcinoma | *** |
| A-431 | Cervical carcinoma | ** |
| BxPc3 | Pancreatic carcinoma | * |
| HeLa | Cervical adenocarcinoma | ***** |
| 293 | Transformed kidney | ***** |
| 143B (tk-) | Osteosarcoma | ***** |
| A549 | Lung carcinoma | **** |
| ES-2 | Ovarian carcinoma | ** |
| MDAH 2774 | Ovarian carcinoma | ** |
| ^{a}In vitro cell killing. Monolayers of the individual cell lines were infected with encapsidated replicons at a multiplicity of infection of 10 i.u./cell as determined by infection on HeLa-H1 cells. The percentage of cells killed by 40 hour post-infection was noted. The scale represents: ***** (>90% killed); **** (75-90% killed); *** (50-74% killed); ** (25-49% killed); * (<25% killed); 0 (no cell death observed). | | |
| ^{b}Patient tumor cell lines were taken directly from brain tumor surgery patients and were not subjected to greater than five serial passages in tissue culture prior to infection. Because of the variation in number of cells in the clinical samples and clumping behavior at early stages of culture, these cell lines were not scored on the scale used for the tissue culture lines. Multiplicities of infection in the assays ranged from 10 to 100 i.u./cell. In each case, >25% of | | |
| # the cells showed cytopathic effects associated with replicon infection (designated as “Y” on the table). | | |

^{c}The 99040123, 01011015, and 010201010 cell lines were infected immediately after recovery from the patient and dispersion of the cells, with no serial passage of the cells in tissue culture.

### Example 4

### In vivo Infection of Transplanted Glioma Cell Lines with Replicons

In vitro studies demonstrated that recombinant replicon vectors could be used to infect glioma cell lines in tissue culture (Table 2). However, to address the possibility that cells susceptible to replicon infection in vitro may become refractory to infection in vivo, the following study was performed.

Human glioma cells grown in scid mice were tested for susceptibility to replicon infection. Previous studies had demonstrated that intracranial implantation of human malignant glioma cells, D54 MG, into scid mice results in tumor growth and, if not treated, will lead to death of the animal (Andreansky S et al., 1996, Proc. Natl. Acad. Sci. USA 93:11313-11318.).

Scid mice were implanted intracranially with D54-MG tumor cells. A time course of intratumoral gene expression in vivo was investigated by injection of encapsidated replicons encoding human interleukin-6 (h-IL6) into D54-MG tumors implanted intracranially in scid mice. After 14 days of tumor growth, the tumors were directly injected with PBS (animals 1, 4, 7, 10, 13) or with 10⁷ of encapsidated replicons which express human IL-6 (animals 2, 3, 5, 6, 8, 9, 11, 12, 14, 15). The animals were sacrificed after either 5 hours (animals 1-3), 8 hours (animals 4-6), 16 hours (animals 7-9), 24 hours (animals 10-12), or 48 hours (animals 13-15), and forebrain and tumor tissue from the right hemisphere and adjacent portions of the left hemisphere were collected. The forebrain tissue was recovered in equivalent amounts from each animal around and including the primary tumor mass present at the injection site. The tissues were homogenized in equivalent volumes of buffer and detergent and lysed by sonication. Equivalent volumes of the tumor/brain tissue homogenates were then assayed for concentration of h-IL6 by using a commercially available ELISA assay kit (R&D Systems).

As seen in FIG. 3, expression of hIL-6 could be detected at very low levels by 5 hours post-injection, and increased with a peak of expression at 16 hours after injection when hIL-6 levels were as high as 15,000 pg/mL of tissue homogenate. By 48 hours post-injection, only background levels of hIL-6 were detected. Similar kinetics of luciferase activity were observed when a replicon encoding luciferase was substituted for that encoding hIL-6. Taken together, these results indicate that the replicon vectors are capable of initiating an infection/expression cycle in vivo that closely mimics that seen for in vitro infection of tumor cells.

### Example 5

### Scope of in vitro Infection of Transplanted Glioma Cell Lines with Replicons

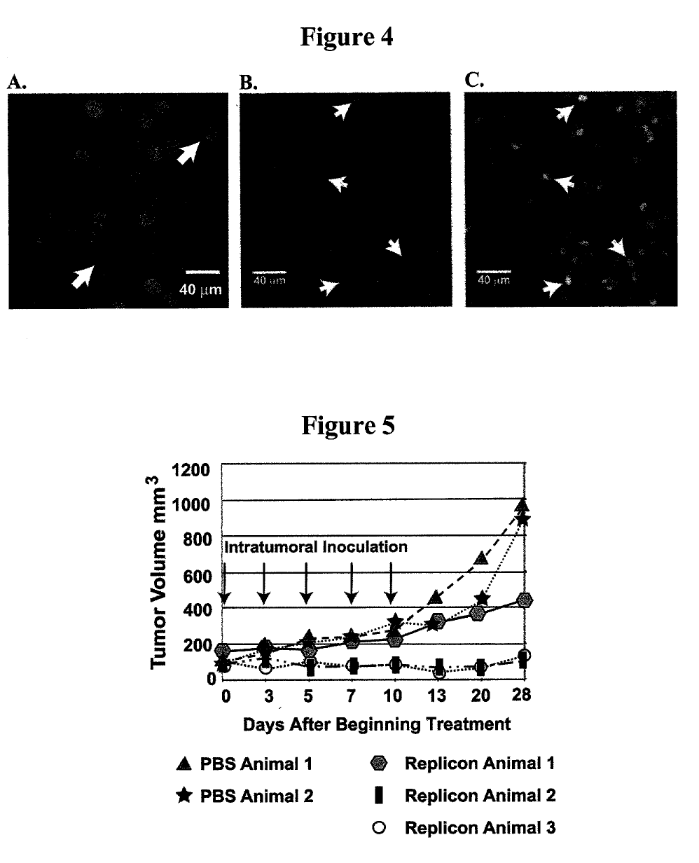

The use of replicons as an oncolytic agent depends on their capacity to infect a significant number of cells following inoculation. It is thus desirable to design replicons to infect as many cells as possible following inoculation. A replicon encoding GFP was used to evaluate the distribution of in vivo infected cells. Pilot experiments indicated that infection of cells in vivo with this replicon construct results in the production of functional GFP with kinetics similar to proteins expressed from other replicons with maximum fluorescence approximately 8-12 hours post infection. For the experiment, human D54-MG malignant glioma cells were allowed to adhere to a coverslip and then infected with 10⁷ i.u./cell of replicons encoding GFP. As illustrated in FIG. 4A, sections displayed numerous tumor cells with vivid fluorescence, indicating widespread infection with GFP replicons. Some of the cells showed cytopathic effects such as rounding, consistent with other observations of replicon-infected D54-MG tumor cells.

To investigate the cell killing effect further, D54-MG cells were infected with replicons encoding GFP at an MOI of 0.3 infectious units per cell, so that the monolayer would contain both infected and uninfected cells. Briefly, D54-MG human glioma cells were grown on glass cover slips (MatTek Corp., Ashland, Mass.) that had been coated with type IV human placental collagen (Sigma) and were infected with encapsidated replicons encoding GFP at a multiplicity of infection of 0.3 i.u. per cell or left uninfected. After 16 hours of infection at 37° C., the monolayers were incubated with Hoechst 33258 Trihydrochloride at a concentration of 20 μg/mL diluted in complete DMEM for one hour, followed by a brief wash in PBS. The stained cells were viewed by using a Leica DIMRBE confocal microscope equipped with a Coherent Enterprise II Inovq ultraviolet laser. The nuclei of the stained cells were visualized for properties associated with apoptosis versus necrosis that are characteristic of the Hoechst stain; that is, apoptotic nuclei are fragmented and condensed into bright clumps, whereas necrotic nuclei appear lightly stained and diffuse due to the extracted nucleoplasm. The staining pattern revealed a substantial number of condensed, brightly staining nuclei, consistent with cells undergoing apoptosis (FIG. 4B). The cells were also viewed for green fluorescence, indicative of expression of GFP in the replicon-infected cells. The image of green fluorescing cells was merged with the image of Hoechst stained nuclei to determine whether a correspondence existed between green-fluorescing cells and nuclei displaying apoptotic characteristics (FIG. 4C). Many of the cells showed characteristics consistent with apoptosis such as nuclear condensation and brighter staining (FIG. 4B, white arrows) and also expressed GFP (FIG. 4C).

### Example 6

### Replicons Inhibit Growth of Human Tumors Transplanted into Scid Mice

The capacity for replicons to specifically infect and kill glioma cells in vivo indicates that these vectors are useful in glioma therapy. This was further demonstrated in vivo using D54-MG gliomas implanted in scid mice. Cells of this tumor line were implanted in the flanks (hindlegs) or intracerebrally in the right caudate nucleus of scid mice as previously described (Andreansky S et al., 1996, 1997, 1998). For flank tumor implants, 2×10⁶ D54-MG cells were resuspended in PBS, pH 7.2 (100 μL per flank implant) and were injected subcutaneously into the right hindleg of the animals. The flank tumors were allowed to grow to 60-100 mm³ in volume as determined by caliper measurement of the length and width of the flank tumors prior to treatments. 1×10⁷ i.u. of encapsidated replicons encoding GFP resuspended in 100 μL of PBS were injected into the flank tumors at the indicated times (or PBS alone for control animals), and tumor sizes were monitored for change every 2-3 days by measurement with calipers. The mean tumor sizes for the PBS group (5 mice) and the group receiving replicon treatments (8 mice) were calculated and compared versus time.

For intracranial studies, D54-MG (1×10⁶ cells in 10 μL of DMEM containing 5% methyl cellulose) were implanted 3 mm deep, 2 mm lateral to midline and 1.5 mm anterior to bregma by injection using a Hamilton 250 μL syringe fitted with a 30G one-half inch needle and attached to a stereotaxic headframe. The implanted tumors were allowed to grow for the desired period of time prior to injection with replicons. For injection of replicons, the indicated amounts of replicons resuspended in PBS were injected through the same burrhole in the skull through which tumor cells were delivered, using the same coordinates identified by the stereotaxic headframe. Following injection of replicons, the mice were allowed to recover and were monitored for survival or were sacrificed for histological analyses as indicated. Animals that had become moribund from progressive tumor growth were sacrificed, and their survival time was ended at the date of sacrifice. All surgeries and post-operative care were performed under UAB IACUC guidelines.

Replicons encoding HSV-TK were used to provide the optional ability to enhance tumor reduction by a bystander killing effect. The flank tumors were then directly injected (designated day 0, FIG. 5) with encapsidated replicons (green hexagon, purple rectangle, and yellow circle) or with PBS (blue star and red triangle), followed by subsequent injections at days 3, 5, 7, 10. The changes in size of the tumors were monitored by caliper measurements. Following the last treatment at day 10, tumor growth was monitored for an additional 18 days prior to sacrifice of the animals.

Control mice implanted with D54-MG gliomas and given intratumoral injections of PBS showed a rapid increase in tumor growth during the 28 day observation period (FIG. 5); the experiments were terminated after that time, because the animals succumbed to the tumors. Three animals given replicons exhibited a completely different clinical response. In one, the tumor showed restricted growth, while in the other two, the tumors showed little or no continuous growth (FIG. 5). Importantly, at some time points, the tumors were reduced in size by as much as 60% over the pre-treatment sizes (FIG. 5).

These effects were observed in the absence of prodrug (gancyclovir) administration. However, addition of the prodrug may be useful in treating other types or sizes of tumors.

### Example 7

### Replicons Enhance Survival of Scid Mice Bearing Intracranial Tumors

It is known that poliovirus has a restricted tropism in the brain. The majority of the infection is confined to the motor cortex with little or no involvement of the cerebral cortex (Bodian D, 1949, Am. J. Med. 6:563-578; Ren R et al., 1990, Cell 63:353-362). Administration of wild-type poliovirus via intracranial inoculation results in infection of the motor cortex and clinical symptoms resembling poliomyelitis (Ren R et al., 1990, Cell 63:353-362). In contrast, recent studies indicate that administration of replicons to the brain via intracranial inoculation does not result in morbidity of transgenic mice which express the human poliovirus receptor (Bledsoe et al., 2000, J. Neurovirol. 6:95-105). These results demonstrate that replicons lack the capacity to cause disease when given intracranially (Bledsoe A W et al., 2000, J. Neuro Virol. 6:95-105; Bledsoe A W et al., 2000, Nature Biotechnology 18:964-969).

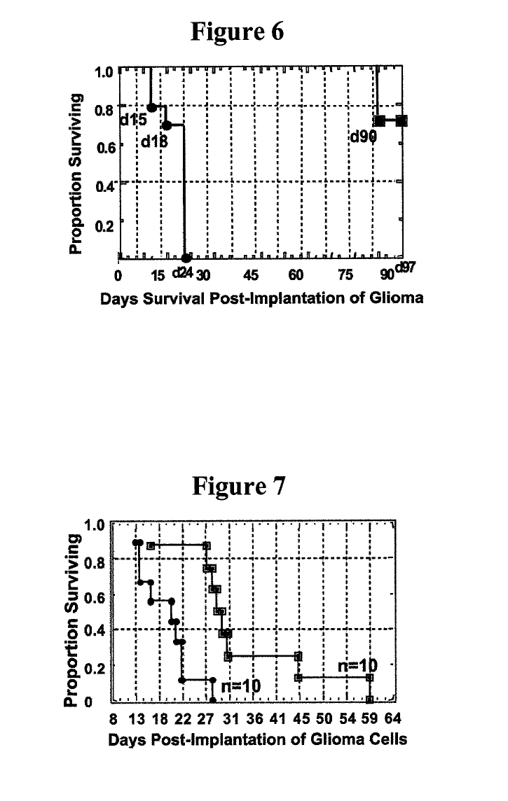

To demonstrate the potential of replicons as an oncolytic agent for gliomas, D54-MG tumor cells were mock infected or treated ex vivo with sufficient replicons to infect all of the tumor cells. The replicon used comprises a nucleic acid encoding tetanus toxin C-fragment. The tumor cells were then implanted intracranially into scid mice which were then followed for evidence of tumor growth, behavioral changes, and survival. As shown by the Kaplan-Meir survival curve in FIG. 6, mice injected with cells that were mock-infected (n=4) rapidly showed signs of tumor development and all died by day 24 post-implantation. However, three mice injected with replicon-treated tumor cells showed no signs of tumor development and were sacrificed at day 97. One mouse given replicon-treated cells died at day 90, and histology confirmed the presence of tumor.

Replicons also effect the survival of mice having established tumors. Intracranial implantation of D54-MG human glioma cells was followed 5 days later with a single injection of either PBS (n=10) or 10⁷ infectious units of encapsidated replicons (n=10). Mice in each group were monitored for for 60 days for survival and sacrificed when moribund. The proportion of surviving mice from each group is shown on a Kaplan-Meier survival plot relative to days post-implantation with the D54-MG cells (FIG. 7). Mice from the PBS control-treated group showed a median survival of 18 days versus 29 days for the replicon-treated group, as determined by log rank test using GB-STAT statistical software, represented a survival increase of 61% (p<0.002).

This study has been repeated using replicons in which other transgenes have been substituted for the capsid (P1) gene (Table 3). Various delivery methods were used. Mice were transplanted with D54-MG tumor cells. Subsequently, mice were administered replicons by single injections, sustained osmotic pump delivery or both. Unless otherwise indicated, single injections were administered immediately following implantation. Unless otherwise indicated, sustained delivery commenced immediately following implantation. In each case, mice treated with replicons had a statistically significant survival advantage over mice treated with the saline control. Replicon controls (“None”) consisted of administration of replicon-free phosphate buffered saline (PBS).

TABLE 3
Replicons enhance survival of tumor bearing animal
irrespective of encoded transgene^{a}.

| Treatment | | | | Total | | |
|---|---|---|---|---|---|---|
| | Sustained | | | Dose | Median | Increase |
| Injection | Delivery | Replicon^{b} | n | (I.U.) | Survival | Over Control |
| 1 at 5 dati | None | None | 6 | | 11 d | |
| | | IL-4 | 6 | 10⁷ | 37.6 d | 242% |
| | | | | | | (p < 0.005) |
| | | TNF- | 6 | 10⁷ | 33.8 d | 207% |
| | | | | | | (p < 0.003) |
| None | Beginning | None | 5 | | 18 d | |
| | 3 dati | IL-4 | 5 | 10⁷ | 27.25 d | 51% |
| | | | | | | (p < 0.04) |
| | | TNF- | 5 | 10⁷ | 28.75 d | 60% |
| | | | | | | (p < 0.04) |
| 1 | Yes | None | 10 | | 16.5 d | |
| | | GM-CSF | 10 | 5 × 10⁷ | 23 d | 40% |
| | | | | | | (p < 0.02) |
| 1 | Yes, over | None | 5 | | 9 d | |
| | 1 week | TNF- | 5 | 5 × 10⁷ | 33.5 d | 272% |
| | | | | | | (p < 0.03) |
| 1 | Yes, over | None | 5 | | 14 d | |
| | 2 weeks | TNF- | 5 | 5 × 10⁷ | 37 d | 164% |
| | | | | | | (p < 0.002) |
| 1 at 4 dati | None | None | 11 | | 21 d | |
| 1 at 8 dati | | PNP | 11 | 2 × (5 × | 32 d | 53% |
| | | | | 10⁷) | | (p < 3 × |
| | | | | | | 10⁻⁶) |

Abbreviations: Number of animals treated (n); days (d); days after tumor implantation (dati); purine nucleoside phosphorylase (PNP).
^{a}Summary of survival analyses in intracranial D54-MG tumor-bearing scid mice using variuos transgene-encoding replicons and delivery methods.
^{b}Replicons encoding various transgenes were constructed as described for the hIL-6 replicon and the GFP replicon.
^{c}Median survival and statistical analyses using the log rank test were calculated by using GB-STAT software.

To demonstrate that this survival advantage was dependent on replicon infection, replicons were exposed to ultra-violet (UV) light prior to injection. UV light is known to destroy poliovirus infectivity. Mice injected with replicons exposed to UV light did not display a survival advantage over controls

Finally, the tumors from the animals given replicons were examined for susceptibility to re-infection following isolation from moribund animals. In all cases, the D54-MG tumors were still 100% susceptible to injection with replicons, indicating the administration of replicons to the tumors in vivo had not resulted in the development of tumors resistant to infection with replicons.

### Example 8

### Histological Analysis of Tumors Treated with Replicons in vivo

Expression of replicon-encoded genes in vivo was verified previously (FIGS. 3 and 4). To further characterize the impact of replicon treatment on the growth of the D54-MG tumors in vivo near the site of injection (forebrain) and at sites within the brain (midbrain sections), scid mice with intracranial D54-MG tumors were injected with 5×10⁷ i.u. of encapsidated replicons encoding GFP. At appropriate time points following injection, the animals were sacrificed and perfused with 4% paraformaldehyde. After post-fixation overnight, the brains were cryo-protected in 30% sucrose and sectioned at 10 mμ with a cryostat. Immunostaining was performed using a rabbit polyclonal antibody against GFP (Invitrogen, Carlsbad, Calif.), followed by an incubation with a biotinylated donkey anti-rabbit secondary antibody (Jackson Immunologicals, West Grove, Pa.) and green Alexa 488 fluorochrome (Molecular Probes, Eugene, Oreg.). A monoclonal primary antibody against human HLA-A,B,C (B.D. Pharmingen, San Diego, Calif.) was used to identify the tumor cells, followed by an incubation with donkey anti-mouse secondary conjugated to an Alexa 568 fluorochrome (Molecular Probes). Sections were visualized using a Leica DIMRBE confocal microscope equipped with an Argon laser for shorter (488 nm) wavelength and a Krypton laser for the longer (568 nm) wavelength signal. All surgeries and post-operative care were performed under UAB IACUC guidelines.

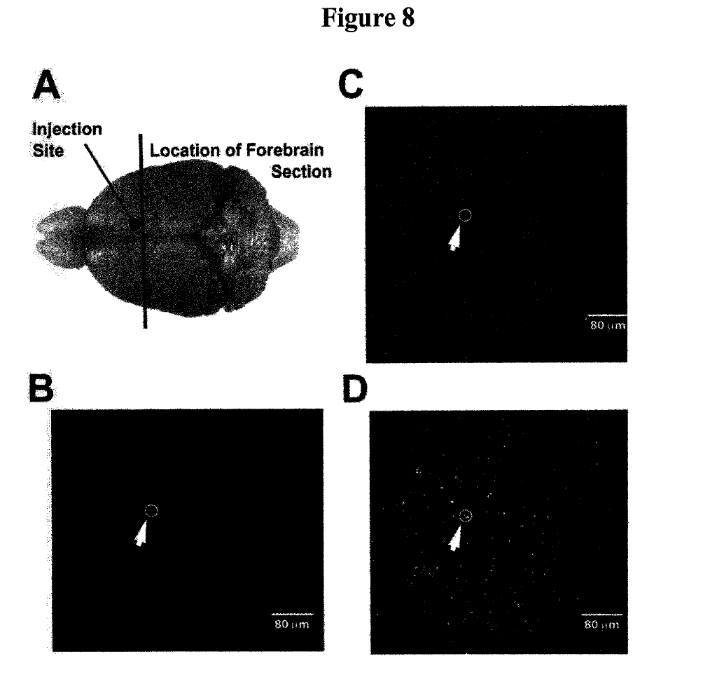

For this study, D54-MG cells were implanted intracranially into scid mice. After 10 days of growth, active (10⁷ infectious units) or UV-inactivated GFP replicons were injected into the same location. FIG. 8A is a photographic representation of a mouse brain with reference points to indicate the site of tumor implantation and intratumoral injection of replicons. The location of the section used for histological analysis in this experiment is also indicated.

A coronal section from the forebrain of a mouse harvested 24 hours post-injection with encapsidated GFP replicons was immunostained with a mouse primary antibody specific for human HLA type II (BD Pharmingen). This antibody stains only the human tumor cells in these sections. A biotinylated donkey anti-mouse secondary antibody (Jackson Immunology, Inc.) cojugated to Alexa 568 fluorochrome (Molecular Probes, Inc.) was applied to the sections. Sections were illuminated with a krypton laser and imaged by confocal laser scanning microscopy (CLSM)(FIG. 8B). Under these conditons the Alexa 568 fluoresces red.

The section pictured in FIG. 8B was further stained with a rabbit polyclonal primary antibody specific for GFP (Invitrogen, Inc.) followed by incubation with a donkey anti-rabbit secondary antibody Jackson Immunology, Inc. conjugated to Alexa 488 fluorochrome (Molecular Probes, Inc.). Immunolabeled GFP was visualized by CLSM under argon laser illumination (FIG. 8C). Green fluorescing cells represent D54-MG tumor cells that were infected by the replicons encoding GFP. Scid mice cells are not susceptible to replicon infection, since they lack the cell surface hPVR required for entry. The images in FIG. 8B and FIG. 8C were merged using the Leica software accompanying the DIRMBE confocal laser microscope (Leica) used for analysis of the tissue sections. Cells which fluoresce both red (human tumor cells) and green (GFP replicon-infected human tumor cells) appear as yellow in color on this panel, e.g. FIG. 8D, circled cell. Red cells on this image represent human tumor cells that were not infected by the GFP replicons.

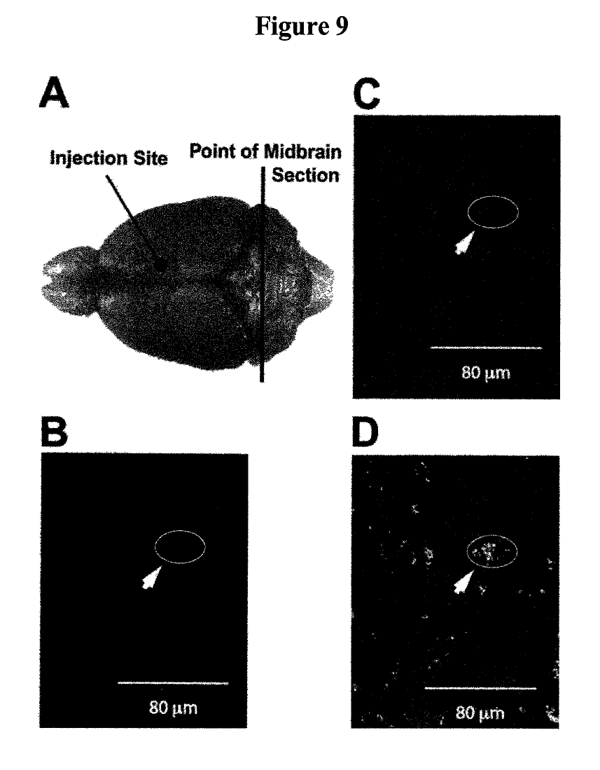

Metastasis of primary tumors frequently results in tumor re-occurrence and pathogenesis. To determine whether replicons are capable of infecting metastases located at sites removed from the initial site of tumor implantation, mid-brain brain sections derived from the mid-brain of animals sacrificed 24 hours post-replicon injection were analyzed (FIG. 9). These mice were treated as described for FIG. 8 above. Immunolabeling and microscopy was performed as described for FIG. 8 above. Tumor metastases (FIG. 9B) infected with GFP replicons (FIG. 9C) were located in these sections. FIG. 9D, a composite of FIGS. 9B and 9C, shows that a significant number of the tumor cells within the metastasis were infected with the replicons, e.g. brightly fluorescent cells within the oval. This indicates that replicons delivered to the forebrain were capable of diffusing to distant regions of the brain and infecting human tumor cells there.

Visual inspection of the tumor sections clearly showed a substantial reduction in the amount of tumor cells present in the GFP replicon-treated brain tissues versus those treated with the UV-inactivated replicons at all time points The animals treated with replicons appeared healthier and more active than the animals given the inactivated replicons, even within one to three days following injection. These observations were consistent with the longer survival of tumor-bearing animals treated with replicons observed in our survival studies (Table 3).

In addition, the sections were analyzed with a commercially available kit to detect apoptosis by TDT-mediated dUTP nicked end labeling (TUNEL) assay. Apoptosis has been proposed as a mechanism by which poliovirus (and many other viruses) are capable of causing the death of cells. Therefore, it seemed plausible that the replicons could be causing an oncolytic effect through induction of the apoptotic pathway. While control sections treated with DNase I were positive for the TUNEL assay which detects DNA fragmentation, apoptotic tumor cells were not detected in the replicon infected tissues. This data should not, however, be construed to limit the scope of this invention to oncolysis by non-apoptotic mechanisms. Replicons may destroy other cell types by apoptosis. Moreover, replicons may destroy D54-MG cells by apoptosis under certain conditions.

### Example 9

### Treatment of Patient Tumor Cells

Replicons are oncolytic in a variety of primary CNS tumors. Tumors excised from patients were trypsinized and applied to tissue culture plates. Cells which adhered to the plate were exposed to luciferase replicons. Results of subsequent luciferase activity and cell death analysis are summarized in Table 2.

### Example 10

### Replicons Gain Entry to Tumor Cells via Interaction with CD155

A major determinant of susceptibility of tumor cells to replicon infection is the presence of the hPVR (also known as CD155) on the surface of the cells (Mendelsohn C L et al., 1989, Cell 56:855-865; Ren R et al., 1990, Cell 63:353-362). The human poliovirus receptor has been well characterized, and its expression on the surface of cells has been shown to be required for infection by wild-type poliovirus (Mendelsohn C L et al., 1989, Cell 56:855-865). According to the invention, an interaction between replicons and CD155 occurs in order for the cancer cells to become infected and express proteins encoded by the RNA genome. This requirement has been demonstrated for eight human cancer cell lines of various tissue origins by performing an antibody inhibition assay using a commercially available monoclonal antibody (MAb) that specifically recognizes CD155 (NeoMarkers, Inc., Fremont, Calif.). This MAb (designated Clone D171) has been well characterized in the literature and has been shown to compete with wild-type poliovirus for binding to the cell surface (Nobis P et al., 1985, J. Gen Virol 66(Pt 12):2563-9). The MAb has been shown to only bind cells that also bind poliovirus; these binding activities are not separable.

For the antibody inhibition experiment, various human cancer cell monolayers were incubated with an anti-CD155 MAb at a series of dilutions from 0 ng/mL to 5000 ng/mL for one hour. After the one-hour antibody adsorption, encapsidated replicons encoding GFP were added to the cell monolayers at a multiplicity of infection of 5 infectious units per cell (MOI=5). The infections were allowed to proceed for 24 hours, and then green fluorescent cells were counted by visualization of fluorescence under ultraviolet light. The number of infected cells was plotted for each cell line as a percentage relative to the control cells in each group that were not exposed to the anti-CD155 antibody.

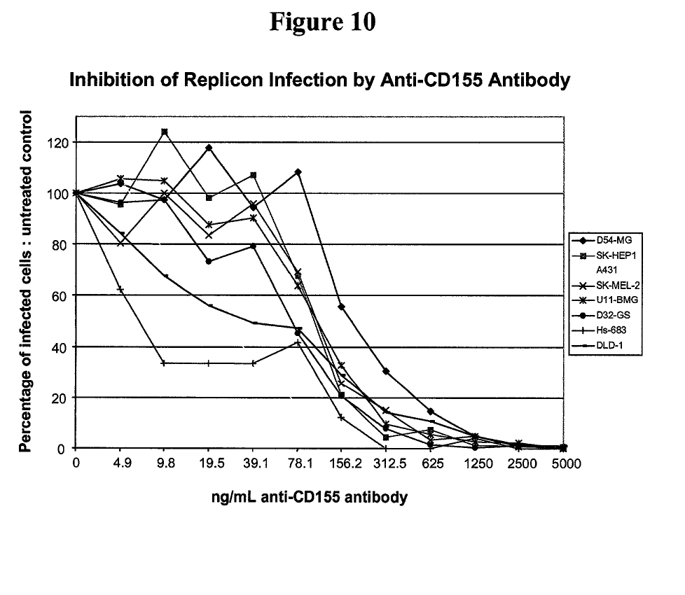

Results are shown in FIG. 10. Human cancer cell lines were incubated with a MAb specific for CD155 at various dilutions for one hour, followed by infection with encapsidated replicons encoding GFP at an MOI=5. The number of cells used in the assay varied between cell lines depending on their different growth characteristics, but in general ranged between 100,000 to 300,000 cells. After a 24 hour incubation, the number of green cells in each culture was counted by visualization of fluorescence under ultraviolet light. The number of green cells at each dilution of antibody was plotted as a percentage relative to the control well for each cell line that was not exposed to antibody prior to infection with replicons. The cell lines tested included D54-MG (malignant glioma), SK-Hep1 (lung adenocarcinoma), A-431 (cervical adenocarcinoma), SK-MEL-2 (melanoma), U11-BMG (glioblastoma multifonne), D32-GS (gliosarcoma), Hs-683 (anaplastic glioma), and DLD-1 (colon carcinoma).

In each case, the anti-CD155 MAb inhibited infection of the cells by the encapsidated GFP replicons. A control cell line (BHK, baby hamster kidney) was not infected by the replicons, whereas BHK cells stably transfected with the gene encoding CD-155 was infected by the GFP replicons In addition, infection of the BHK cells that express CD-155 was blocked by anti-CD155 antibody In the case of the human cancer cell lines, variation in the concentration of antibody required for inhibition to occur on the various cell lines was observed. The D54-MG malignant glioma cell line required the highest antibody concentration for inhibition to occur (233.9 ng/mL for 50% inhibition). This cell line was also the most sensitive to replicon infection at the dose used (MOI 5) in this assay, yielding more than twice as many infected cells (expressed as a percentage of total cells in the well) after the 24 hour incubation than all of the other cancer cell lines tested. The antibody treatment clearly inhibited an early step in infection by the replicons, as D54-MG cells that were exposed to GFP replicons for one hour first and then treated with the anti-CD 155 antibody showed the same level of infection as untreated D54-MG cells The other cell lines required an antibody concentration of 50% or less than that of D54-MG for 50% inhibition of infection by the GFP replicons. These results demonstrate that the interaction of replicons with the CD155 receptor on the surface of the cancer cells is required for infection, and suggests that one of the determinants in variation of sensitivity to infection by replicons may be amount of CDl155 on the surface of the cells.

### Example 11

### Construction of Replicons

The replicon which encodes green fluorescent protein (GFP) was constructed by using previously described methods (Porter D C et al., 1998; Jackson C A et al., 2001). Briefly, the gene segment encoding GFP (Clonetech, Palo Alto, Calif.) was amplified by polymerase chain reaction, and the resulting PCR product was subcloned into a plasmid containing the replicon cDNA; this replicon cDNA contains an in-frame deletion of the poliovirus capsid gene between the VP2/VP3 capsid gene junction and the remainder of the VP3 and VP 1 capsid proteins, except for sequences encoding the last seven amino acids at the C-terminus of VP 1. The GFP gene fragment was inserted into this plasmid between a unique Xho I site introduced at the VP2/VP3 junction and a unique Sna BI site at 3359. At the 5′ end of GFP, a 19 amino acid sequence encoding a self-cleaving peptide derived from foot and mouth disease virus (FMDV) was inserted (Mattion N M et al., 1996). Translation of the peptide results in autocatalytic cleavage, leaving a proline amino acid at the amino terminus of GFP (FIG. 1A). The autocatalytic activity of the 2A protease liberates the GFP protein COOH-terminus at the natural junction of VP1 and 2A, which is maintained in the replicon RNA genome. Expression of GFP was confirmed by immunoprecipitation of metabolically labeled protein with antibodies specific for GFP, as well as direct visualization of GFP-mediated fluorescence in cells viewed under ultraviolet fluorescence (FIG. 1B).

The replicon that encodes human IL-6 (h-IL6) was constructed in a similar manner by insertion of the gene encoding h-IL6 (complete cDNA purchased from R&D Systems, Minneapolis, Minn.) in place of the capsid sequences deleted from the replicon cDNA. The complete h-IL6 gene was amplified by polymerase chain reaction and was subcloned into the replicon cDNA plasmid as described previously by using Aho I (5′ end) and Sna BI (3′ end) restriction endonuclease sites incorporated at the ends of the amplification primers. The sequences of the primers used for amplification of the h-IL6 gene were 5′-CTC-GAG-ATG-AAC-TCC-TTC-TCC-3′ (SEQ ID NO:1) and 5′-TAC-GTA-CTA-CAT-TTG-CCG-AAG-3′ (SEQ ID NO:2). Expression of h-IL6 was confirmed by immunoprecipitation of metabolically radiolabeled proteins with an antibody specific for h-IL6 (R&D Systems), as well as by analysis of lysates from cells infected with the replicons by using a commercially available ELISA kit specific for h-IL6 detection (R&D Systems). Additional replicons encoding HSV-TK or other proteins described in Table 2 will be described elsewhere.

### Example 12

### Tissue Culture Cells and Viruses

Encapsidated replicons were grown in HeLa-H1 cells which were maintained in Dulbecco's Modified Eagle Medium (DMEM, Life Technologies of Rockville, Md.) supplemented with 5% fetal bovine serum (Life Technologies) and 1% Antibiotic/Antimycotic (Life Technologies). The modified vaccinia virus that expresses the poliovirus capsid protein was grown in chicken embryo fibroblasts and maintained in DMEM supplemented with 10% fetal bovine serum. Some tumor cell lines were purchased from American Type Culture Collection (Rockville, Md.) for this study (IMR-32, SK-MEL-28, BT20, HT1080, DLD-1, SK-Hep1, 293, 143B-TK⁻, A549, ES-2, and MDAH2774); other lines have been grown at the University of Alabama at Birmingham for several years in the laboratory of Dr. G. Yancey Gillespie (D54-MG, U251-MG, U373-MG, D32GS, SK-N-MC, CH-157-MN, U118-MG, Hs-683, SK-MEL-2, SK-MEL-21, SQ-20-B, A-431, and BxPc3). Primary tumor cells from patients undergoing surgery for brain tumors had been subjected to less than five serial passages prior to infection with encapsidated replicons. All tumor cell lines and patient tumor cells were maintained in DMEM-F12 (Life Technologies) supplemented with 10% FBS. Patient tumor lines were received and used for experiments under approval of the UAB IRB.

### Example 11

### Introduction of Replicons to Cells by Transfection Methods

Replicon RNA genomes can also be delivered to cancer cells independent of either the poliovirus capsid or the CD 155 receptor present on the cell surface. The direct oncolytic activity of the replicons is inherent to the replicating RNA genome. This activity has been demonstrated by performing in vitro transfection of human cancer cell lines with replicon RNA genomes encoding GFP. These RNA genomes were transcribed from cDNA templates in vitro by using bacteriophage T7 RNA polymerase. The in vitro transcribed replicon RNA molecules were complexed with either liposomes (Lipofectin transfection reagent, GIBCO/BRL) or with polyethylenimine (PEI) and then incubated with either HeLa cells (human cervical carcinoma) or A549 cells (human lung carcinoma cells). After an overnight incubation at 37° C., cells transfected with the GFP replicon RNA genomes displayed the same green fluorescence and cytopathic effects (e.g., rounding of the cells) observed when the cells were infected with encapsidated replicons encoding GFP. Upon further incubation, the transfected cells lysed, as evidenced by detachment from the plate, within 24-48 hours. These results are consistent with observations made of human cancer cells infected with encapsidated replicons via the CD 155 receptor interaction and demonstrate that the oncolytic activity associated with the replicons is inherent to the RNA genome itself.

### Example 12

### Safety

In preferred embodiments of the invention, replicons have little or no deleterious effects on normal tissue. Previous studies have established a clear safety profile for the administration of replicons in the periphery as well as in the brain and central nervous system (Bledsoe A W et al., 2000, J. Neuro Virol. 6:95-105; Bledsoe A W et al., 2000, Nature Biotechnology 18:964-969). Transgenic mice expressing the human poliovirus receptor have been shown to be extremely susceptible to poliovirus administered by a variety of routes including peripherial administration and direct CNS delivery (Ren R. et al, 1990, Cell 63:353-362; Bledsoe A W et al., 2000, J. Neuro Virol. 6:95-105; Deatly A M et al., 1999, Virology 255:221-227). These transgenic mice are so susceptible to wild type poliovirus that as little as 100 pfu administered intraspinally results in death (Bledsoe A W et al., 2000, J. Neuro Virol. 6:95-105; Deatly A M et al., 1999, Virology 255:221-227).

These transgenic animals were selected for analysis of replicon safety. Administration of replicons encoding proteins such as GFP or luciferase at 10,000 fold greater amounts than a wild type poliovirus lethal dose has not resulted in any deleterious effects following direct intraspinal administration. Animals have been monitored for behavioral abnormalities as well as a complete histological analysis. In addition, some trangenic mice have been subjected to up to thirteen sequential CNS administrations of replicons with no deleterious effects observed. Thus, repeated replicon administration to normal cells, even those with cell surface hPVR, does not result in deleterious effects.

References

The references cited throughout the application and hereinafter are incorporated in their entirety by reference.

Andreansky S S et al., 1996, “The application of genetically engineered herpes simplex viruses to the treatment of experimental brain tumors”, Proc. Natl. Acad. Sci. USA. 93:11313-11318.

Andreansky S et al., 1997, “Evaluation of genetically engineered herpes simplex viruses as oncolytic agents for human malignant brain tumors”, Cancer Research 57:1502-1509.

Andreansky S et al., 1998, “Treatment of intracranial gliomas in immunocompetent mice using herpes simplex viruses that express murine interleukins” Gene Therapy 5:121-130.

Ansardi D C et al. Characterization of poliovirus replicons encoding carcinoembryonic antigen. Cancer Research 54, 6359-6364 (1994).

Ansardi, D. C., Porter, D. C. & Morrow, C. D. Complementation of a poliovirus defective genome by a recombinant vaccinia virus which provides P1 capsid precursor in trans. J. Virol. 67, 3684-3690 (1993a).

Ansardi D C et al., 2001, “RNA replicons derived from poliovirus are directly oncolytic for human tumor cells of diverse origin” Cancer Research 61:8470-8479.

Asano, T. Treatment of human cancer with mumps virus. Cancer 34, 1907-1928 (1974).

Barba Det al., 1994, “Development of anti-tumor immunity following thymidine kinase-mediated killing of experimental brain tumors”, Proc. Natl. Acad. Sci. USA 91:4348-4352 1994.

Barco, A., Feduchi, E. & Carrasco, L. Poliovirus protease 3C(pro) kills cells by apoptosis. Virology 266, 352-360 (2000).

Blaser, M. J. Linking Helicobacter pylori to gastric cancer. Nature Medicine 6, 376-377 (2000).

Bledsoe A W et al., 2000, “Cytokine production in motor neurons by poliovirus replicon vector gene delivery”, Nature Biotechnology 18:964-969.

Bledsoe A W et al., 2000, “Targeted foreign gene expression in spinal cord neurons using poliovirus replicons”, J. Neuro Virol. 6:95-105.

Bleyer, W. A. & Byrne, T. N. Leptomeningeal cancer in leukemia and solid tumors. Curr. Probl. Cancer 12, 181-238 (1988).

Bodian D, 1949, “Histopathological basis of clinical findings in poliomyelitis”, Am. J. Med 6:563-578.

Cassel, W. A. & Garrett, R. E. Newcastle Disease Virus as an antineoplastic agent. Cancer 18, 863-868 (1963).

Cassel, W. A., Murray, D. R. & Phillips, H. S. A phase II study on the postsurgical management of stage II malignant melanoma with a Newcastle Disease Virus oncolysate. Cancer 52, 856-860 (1983).

Das S et al., 1993, “Identification of the cleavage site and determinants required for poliovirus 3Cpro-catalyzed cleavage of human TATA-binding transcription factor TBP”, J. Virol. 67:3326-3331.

DeAngelis, L. M. Current diagnosis and treatment of leptomeningeal metastasis. J. Neuro. Onc. 38, 245-252 (1998).

Deatly A M et al., 1999, “Poliomyelitis in Intraspinally Inoculated Poliovirus Receptor Transgenic Mice”, Virology 255:221-227.

Goldstaub D et al., 2000, “Poliovirus 2A protease induces apoptotic cell death”, Mol. Cell Biol. 20(4):1271-1277.

Gromeier M et al., 2000, “Intergeneric poliovirus recombinants for the treatment of malignant glioma”, Proc. Natl. Acad. Sci. USA 97(12):6803-6808.

Gromeier M et al., 2000, “Expression of the human poliovirus receptor/CD155 gene during development of the central nervous system: Implications for the pathogenesis of poliomyelitis”, Virol. 273:248-257.

Hildebrand, J. Prophylaxis and treatment of leptomeningeal carcinomatosis in solid tumors of adulthood. J. Neuro. Onc. 38, 193-198 (1998).

Hughes B W et al., 1995, “Bystander killing of melanoma cells using the human tyrosinase promoter to express the Escherichia coli purine nucleoside phosporylase gene”, Caner Res. 55:3339-3345.

Jackson C A et al., 2001, “Repetitive intrathecal injections of poliovirus replicons result in gene expression in neurons of the central nervous system without pathogenesis” Hum Gene Ther. 12(15):1827-41.

Khuri F R et al. A controlled trial of intratumoral ONYX-015, a selectively-replicating adenovirus, in combination with cisplatin and 5-fluorouracil in patients ith recurrent head and neck cancer. Nature Med. 6, 879-885 (2000).

Kim J H et al., 1994, “Selective enhancement by an antiviral agent of the radiation-induced cell killing of human glioma cells transduced with the HSV-TK gene”, Cancer Res. 54:6053-6055.

Klatzmann D et al., 1996, “Gene therapy for glioblastoma in adult patients: safety and efficacy evaluation of an in situ injection of recombinant retroviruses producing cells carrying the thymidine kinase gene of the Herpes Simplex type 1 virus, to be followed with the administration of ganciclovir”, Human Gene Therapy 7:109-126.

Komblith P K et al., 1993, “The future of therapy for glioblastoma”, Surg. Neurol. 39:538-543.

Lorence, R. M. et al. Complete regression of human fibrosarcoma xenograft after local Newcastle Disease Virus therapy. Cancer Res. 54, 6017-6021 (1994).

Lorence, R. M. et al. Complete regression of human neuroblastoma xenografts in athymic mice after local Newcastle Disease Virus therapy. J. Natl. Can. Inst. 86, 1228-1233 (1994).

Mattion N M et al., 1996, “Foot-and-Mouth disease virus 2A protease mediates cleavage in attenuated Sabin 3 poliovirus vectors engineered for delivery of foreign antigens” Journal of Virology 70(11):8124-8127.

Mendelsohn, C. L., Wimmer, E. & Racaniello, V. R. Cellular receptor for poliovirus: molecular cloning, nucleotide sequence, and expression of a new member of the immunoglobulin superfamily. Cell 56, 855-865 (1989).

Moore, A. E. Viruses with oncolytic properties and their adaptation to tumors. Annals of New York Acd. of Sci, pp. 945-952.

Moore, J. P. & Ho, D. D. Antibodies to discontinuous or conformationally sensitive epitopes on the gp120 glycoprotein of human immunodeficiency virus type 1 are highly prevalent in sera of infected humans. J. Virol. 67, 863-875 (1993).

Mullen C A et al., 1992, “Transfer of the bacterial gene for cytosine deaminase to mammalian cells confers lethal sensitivity to 5-fluorocytosine: a negative selection system”, Proc. Natl. Acad. Sci. (USA) 89:33-37.

Mullen C A et al., 1994, “Tumors expressing the cytosine deaminase gene can be eliminated in vivo wiht 5-fluorocytosine and induce protective immunity to wild-type tumor”, Cancer Res. 54:1503-1506.

Nobis, P., et al., Production of a monoclonal antibody against an epitope on HeLa cells that is the functional poliovirus binding site. J Gen Virol, 1985. 66(Pt 12): p. 2563-9.

Porter D C et al., 1995, “Encapsidation of poliovirus replicons encoding the complete human immunodeficiency virus type 1 gag gene using a complementation system which provides the P1 capsid protein in trans”. J. Virol. 69:1548-1555.

Porter D C et al., 1998, “Demonstration of the specificity of poliovirus encapsidation using a novel replicon which encodes enzymatically active firefly luciferase”, Virology 243(1):1-11.

Porter, D. C., Melsen, L. R., Compans, R. W. & Morrow, C. D. Release of virus-like particles from cells infected with poliovirus replicons which express HIV-1 Gag. J. Virol. 70:2643-2649 (1996).

Racaniello V R et al., 1981, “Cloned poliovirus complementary DNA is infectious in mammalian cells”, Science 214(4542) 916-919.

Reichard, K. W. et al. Newcastle Disease Virus selectively kills human tumor cells. J. of Surg. Res. 52, 448-453 (1992).

Ren R et al., 1990, “Transgenic mice expressing a human poliovirus receptor: a new model for poliomyelitis”, Cell 63:353-362.

Roux P et al., 1989, “A versatile and potentially general approach to the targeting of specific cell types by retroviruses: application to the infection of human cells by means of major histocompatibility complex class I and class II antigens by mouse ecotropic murine leukemia virus-derived viruses”, Proc. Natl. Acad. Sci. USA 86 (23):9079-9083.

Roth, J. A. & Cristiano, R. J. Gene therapy for cancer: what have we done and where are we going? J. Natl. Can. Inst. 89, 21-39 (1997).

Smith, R. R., Huebner, R. J., Rowe, W. P., Schatten, W. E. & Thomas, L. B. Studies on the use of viruses in the treatment of carcinoma of the cervix. Cancer 9, 1211-1218 (1956).

Solecki D et al., 2000, “Identification of a nuclear respiratory factor-1 binding site within the core promoter of the human polio virus receptor/CD55 gene”, J. Biol. Chem. 275:12453-12462.

Solecki D et al., 1999, “Identification and characterization of the cis-acting elements of the human CD155 gene core promoter”, J. Biol. Chem. 274:1791-1800.

Southam, C. M., 1960, “Present status of oncolytic virus studies. The New York Academy of Sciences, 657-673.

Stark W M et al., 1992, “Catalysis by site-specific recombinases” Trends Genet. 8(12):432-439.

Strong J E et al., Coffey, C., Tang, D., Sabinin, P. & Lee, P. W. K. The molecular basis of viral oncolysis; usurpation of the Ras signaling pathway by reovirus. The EMBO J. 17, 3351-3362 (1998).

Taylor, M. W., Cordell, B., Southrada, M. & Prather, S. Viruses as an aid to cancer therapy: Regression of solid and ascites tumors in rodents after treatment with bovine enterovirus. Pro. Natl. Acad. (USA) 68, 836-840 (1971).

Yang W X et al., 1997, “Efficient delivery of circulating poliovirus to the central nervous system independently of poliovirus receptor”, Virol. 229:421-428.

We claim:

1. A method of killing a tumor cell comprising contacting the tumor cell with a replicon such that the replicon is taken up into said tumor cell and said tumor cell is killed.

2. The method of claim 1 wherein the replicon comprises an RNA genome.

3. The method of claim 1 wherein the replicon comprises a DNA genome.

4. The method of claim 1 wherein the replicon is encapsidated.

5. The method of claim 1 wherein the replicon is not encapsidated.

6. The method of claim 1 wherein the replicon kills only the cell contacted and in which it is taken up.

7. The method of claim 1 wherein the tumor cell is killed in vivo.

8. The method of claim 1 wherein the tumor cell is killed in vitro.

9. The method of claim 1 wherein the tumor cell is a central nervous system tumor cell.

10. The method of claim 1 wherein the tumor cell is a non-central nervous system tumor cell.

11. The method of claim 9 wherein the central nervous system tumor cell is selected from the group consisting of an astrocytoma cell, an anaplastic glioma cell, an anaplastic astrocytoma cell, an ependymoma cell, a gliosarcoma cell, a glioblastoma multiforme cell, a malignant glioma cell, a melanoma cell, a meningioma cell, a neuroblastoma cell, an oligodendoglioma cell, and a pilocytic astrocytoma cell.

12. The method of claim 10 wherein the non-central nervous system tumor cell is selected from the group consisting of a breast cancer cell, a cervical carcinoma cell, a cervical adenocarcinoma cell, a colon cancer cell, a fibrosarcoma cell, a lung adenocarcinoma cell, a lung carcinoma cell, an osteosarcoma cell, an ovarian carcinoma cell, a pancreatic carcinoma cell, a squamous cell carcinoma cell, and a transformed kidney cell.

13. The method of claim 1 further comprising contacting said tumor cell with an agent that increases the amount of poliovirus receptor on the surface of said tumor cell.

14. The method of claim 13 wherein said agent is selected from the group consisting of hemin and retinoic acid.

15. The method of claim 4 further comprising contacting an encapsidated oncolytic replicon with CD155 on the surface of the target cell.

16. The method of claim 1 wherein the replicon lacks a heterologous nucleic acid.

17. The method of claim 1 wherein the replicon comprises at least one heterologous nucleic acid.

18. The method of claim 17 wherein at least one heterologous nucleic acid is selected from the group consisting of a transgene, a site-specific mutation, a restriction site, a site-specific recombination site, and an expression control sequence.

19. The method of claim 18 wherein the heterologous nucleic acid is a transgene.

20. The method of claim 19 wherein the transgene encodes a cytotoxic protein.

21. The method of claim 20 wherein the cytotoxic protein is selected from the group consisting of urokinase, tumor neucrosis factorα, and interleukin-4.

22. The method of claim 19 wherein the transgene encodes a prodrug converting protein.

23. The method of claim 22 wherein the prodrug converting protein is selected from the group consisting of herpesvirus thymidine kinase, purine nucleoside phosphorylase, and cytosine deaminase.

24. The method of claim 19 wherein the transgene encodes a protein selected from the group consisting of luciferase, green fluorescence protein, β-glucuronidase, IL-6, and granulocyte/macrophage colony-stimulating factor.

25. The method of claim 19 wherein the transgene encodes an immunogen.

26. The method of claim 25 wherein the inmmunogen is selected from the group consisting of hepatitis B surface antigen, influenza virus hemaglutinin and neuraminidase, human immunodeficiency viral protein, respiratory syncycial virus G protein, a bacterial antigen, a chimeric non-poliovirus gene, a B cell epitope, and a T cell epitope.

27. The method of claim 26 wherein the human immunodeficiency viral protein is selected from the group consisting of gag, pol, and env.

28. A method of inhibiting the growth of a tumor comprising contacting the tumor with a replicon such that the replicon is taken up into said tumor and said growth of said tumor is inhibited.

29. The method of claim 28 wherein the replicon comprises an RNA genome.

30. The method of claim 28 wherein the replicon comprises a DNA genome.

31. The method of claim 28 wherein the replicon is encapsidated.

32. The method of claim 28 wherein the replicon is not encapsidated.

33. The method of claim 28 wherein the tumor is a central nervous system tumor.

34. The method of claim 28 wherein the tumor is a non-central nervous system tumor.

35. The method of claim 33 wherein the central nervous system tumor is selected from the group consisting of astrocytoma, anaplastic glioma, anaplastic astrocytoma, ependymoma, gliosarcoma, glioblastoma multiforme, malignant glioma, melanoma, meningioma, neuroblastoma, oligodendoglioma, and pilocytic astrocytoma.

36. The method of claim 34 wherein the non-central nervous system tumor is selected from the group consisting of a breast tumor, cervical carcinoma, cervical adenocarcinoma, a colon tumor, fibrosarcoma, lung adenocarcinoma, lung carcinoma, osteosarcoma, ovarian carcinoma, pancreatic carcinoma, squamous cell carcinoma, and a kidney tumor.

37. The method of claim 28 further comprising contacting said tumor with an agent that increases the amount of poliovirus receptor on the surface of the cells of said tumor.

38. The method of claim 37 wherein said agent is selected from the group consisting of hemin and retinoic acid.

39. The method of claim 31 further comprising contacting an encapsidated oncolytic replicon with CD155 on the surface of the tumor cell.

40. The method of claim 28 wherein the replicon lacks a heterologous nucleic acid.

41. The method of claim 28 wherein the replicon comprises at least one heterologous nucleic acid.

42. The method of claim 41 wherein at least one heterologous nucleic acid is selected from the group consisting of a transgene, a site-specific mutation, a restriction site, a site-specific recombination site, and an expression control sequence.

43. The method of claim 42 wherein the heterologous nucleic acid is a transgene.

44. The method of claim 43 wherein the transgene encodes a cytotoxic protein.

45. The method of claim 44 wherein the cytotoxic protein is selected from the group consisting of urokinase, tumor neucrosis factor-α, and interleukin-4.

46. The method of claim 43 wherein the transgene encodes a prodrug converting protein.

47. The method of claim 46 wherein the prodrug converting protein is selected from the group consisting of herpesvirus thymidine kinase, purine nucleoside phosphorylase, and cytosine deaminase.

48. The method of claim 43 wherein the transgene encodes a protein selected from the group consisting of luciferase, green fluorescence protein, β-glucuronidase, IL-6, and granulocyte/macrophage colony-stimulating factor.

49. The method of claim 43 wherein the transgene encodes an immunogen.

50. The method of claim 48 wherein the inmmunogen is selected from the group consisting of hepatitis B surface antigen, influenza virus hemaglutinin and neuraminidase, human immunodeficiency viral protein, respiratory syncycial virus G protein, a bacterial antigen, a chimeric non-poliovirus gene, a B cell epitope, and a T cell epitope.

51. The method of claim 50 wherein the human immunodeficiency viral protein is selected from the group consisting of gag, pol, and env.

52. A method of introducing an replicon into a tumor cell having CD155 on its surface comprising contacting an encapsidated replicon with said CD 155 under conditions that permit uptake of the replicon into the cell.

53. The method of claim 52 wherein the replicon comprises an RNA genome.

54. The method of claim 52 wherein the replicon comprises a DNA genome.

55. The method of claim 52 wherein said contact is in vivo.

56. The method of claim 52 wherein said contact is in vitro.

57. The method of claim 52 wherein the tumor cell is a central nervous system tumor cell.

58. The method of claim 52 wherein the tumor cell is a non-central nervous system tumor cell.

59. The method of claim 57 wherein the central nervous system tumor cell is selected from the group consisting of an astrocytoma cell, an anaplastic glioma cell, an anaplastic astrocytoma cell, an ependymoma cell, a gliosarcoma cell, a glioblastoma multiforme cell, a malignant glioma cell, a melanoma cell, a meningioma cell, a neuroblastoma cell, an oligodendoglioma cell, and a pilocytic astrocytoma cell.

60. The method of claim 58 wherein the non-central nervous system tumor cell is selected from the group consisting of a breast cancer cell, a cervical carcinoma cell, a cervical adenocarcinoma cell, a colon cancer cell, a fibrosarcoma cell, a lung adenocarcinoma cell, a lung carcinoma cell, an osteosarcoma cell, an ovarian carcinoma cell, a pancreatic carcinoma cell, a squamous cell carcinoma cell, and a transformed kidney cell.

61. The method of claim 52 further comprising contacting said tumor cell with an agent that increases the amount of poliovirus receptor on the surface of said tumor cell.

62. The method of claim 61 wherein said agent is selected from the group consisting of hemin and retinoic acid.

63. The method of claim 52 wherein the replicon lacks a heterologous nucleic acid.

64. The method of claim 52 wherein the replicon comprises at least one heterologous nucleic acid.

65. The method of claim 64 wherein at least one heterologous nucleic acid is selected from the group consisting of a transgene, a site-specific mutation, a restriction site, a site-specific recombination site, and an expression control sequence.

66. The method of claim 65 wherein the heterologous nucleic acid is a transgene.

67. The method of claim 66 wherein the transgene encodes a cytotoxic protein.

68. The method of claim 67 wherein the cytotoxic protein is selected from the group consisting of urokinase, tumor neucrosis factor-α, and interleukin-4.

69. The method of claim 66 wherein the transgene encodes a prodrug converting protein.

70. The method of claim 69 wherein the prodrug converting protein is selected from the group consisting of herpesvirus thymidine kinase, purine nucleoside phosphorylase, and cytosine deaminase.

71. The method of claim 66 wherein the transgene encodes a protein selected from the group consisting of luciferase, green fluorescence protein, β-glucuronidase, IL-6, and granulocyte/macrophage colony-stimulating factor.

72. The method of claim 66 wherein the transgene encodes an immunogen.

73. The method of claim 72 wherein the inmmunogen is selected from the group consisting of hepatitis B surface antigen, influenza virus hemaglutinin and neuraminidase, human immunodeficiency viral protein, respiratory syncycial virus G protein, a bacterial antigen, a chimeric non-poliovirus gene, a B cell epitope, and a T cell epitope.

74. The method of claim 73 wherein the human immunodeficiency viral protein is selected from the group consisting of gag, pol, and env.

75. A method of introducing a replicon into a tumor cell comprising contacting an unencapsidated replicon with said tumor cell under conditions that permit uptake of the replicon into the cell.

76. The method of claim 75 wherein the replicon comprises an RNA genome.

77. The method of claim 75 wherein the replicon comprises a DNA genome.

78. The method of claim 75 wherein said contact is in vivo.

79. The method of claim 75 wherein said contact is in vitro.

80. The method of claim 75 wherein the tumor cell is a central nervous system tumor cell.

81. The method of claim 75 wherein the tumor cell is a non-central nervous system tumor cell.

82. The method of claim 80 wherein the central nervous system tumor cell is selected from the group consisting of an astrocytoma cell, an anaplastic glioma cell, an anaplastic astrocytoma cell, an ependymoma cell, a gliosarcoma cell, a glioblastoma multiforme cell, a malignant glioma cell, a melanoma cell, a meningioma cell, a neuroblastoma cell, an oligodendoglioma cell, and a pilocytic astrocytoma cell.

83. The method of claim 81 wherein the non-central nervous system tumor cell is selected from the group consisting of a breast cancer cell, a cervical carcinoma cell, a cervical adenocarcinoma cell, a colon cancer cell, a fibrosarcoma cell, a lung adenocarcinoma cell, a lung carcinoma cell, an osteosarcoma cell, an ovarian carcinoma cell, a pancreatic carcinoma cell, a squamous cell carcinoma cell, and a transformed kidney cell.

84. The method of claim 75 wherein the replicon is comprised in a liposome.

85. The method of claim 75 wherein the replicon is complexed with polyethylenimine.

86. The method of claim 75 wherein the replicon lacks a heterologous nucleic acid.

87. The method of claim 75 wherein the replicon comprises at least one heterologous nucleic acid.

88. The method of claim 87 wherein at least one heterologous nucleic acid is selected from the group consisting of a transgene, a site-specific mutation, a restriction site, a site-specific recombination site, and an expression control sequence.

89. The method of claim 88 wherein the heterologous nucleic acid is a transgene.

90. The method of claim 89 wherein the transgene encodes a cytotoxic protein.

91. The method of claim 90 wherein the cytotoxic protein is selected from the group consisting of urokinase, tumor neucrosis factor-α, and interleukin-4.

92. The method of claim 89 wherein the transgene encodes a prodrug converting protein.

93. The method of claim 92 wherein the prodrug converting protein is selected from the group consisting of herpesvirus thymidine kinase, purine nucleoside phosphorylase, and cytosine deaminase.

94. The method of claim 89 wherein the transgene encodes a protein selected from the group consisting of luciferase, green fluorescence protein, β-glucuronidase, IL-6, and granulocyte/macrophage colony-stimulating factor.

95. The method of claim 89 wherein the transgene encodes an immunogen.

96. The method of claim 95 wherein the inmmunogen is selected from the group consisting of hepatitis B surface antigen, influenza virus hemaglutinin and neuraminidase, human immunodeficiency viral protein, respiratory syncycial virus G protein, a bacterial antigen, a chimeric non-poliovirus gene, a B cell epitope, and a T cell epitope.

97. The method of claim 96 wherein the human immunodeficiency viral protein is selected from the group consisting of gag, pol, and env.

98. An antitumor composition comprising a replicon and a carrier.

99. The antitumor composition of claim 98 wherein the replicon genome is RNA.

100. The antitumor composition of claim 98 wherein the replicon genome is DNA.

101. The antitumor composition of claim 98 wherein the replicon is encapsidated.

102. The antitumor composition of claim 98 wherein the replicon is not encapsidated.

103. The antitumor composition of claim 98 wherein the capsid is selected from the group consisting of a wild type poliovirus capsid, a poliovirus type 1 Mahoney capsid, and a Sabin capsid.

104. The antitumor composition of claim 98 wherein the replicon lacks a heterologous nucleic acid.

105. The antitumor composition of claim 98 wherein the replicon comprises a heterologous nucleic acid.

106. The antitumor composition of claim 98 further comprising a bifunctional complex comprising a replicon-binding element and a cell surface molecule-binding element.

107. The antitumor composition of claim 106 wherein the replicon-binding element is selected from the group consisting of an anti-poliovirus capsid protein and a poliovirus receptor.

108. The antitumor composition of claim 106 wherein the cell surface molecule is selected from the group consisting of folate receptor, transferrin receptor, fibroblast growth factor receptor, epidermal growth factor receptor, c-kit receptor, erythrocyte growth factor receptor, polymeric Ig receptor, erythropoietin receptor, purinoceptor, and a metaloproteinase.

109. The pharmaceutical of claim 106 wherein the cell surface molecule-binding element is selected from the group consisting of a folate receptor ligand, a transferrin receptor ligand, a fibroblast growth factor receptor ligand, an epidermal growth factor receptor ligand, a c-kit receptor ligand, an erythrocyte growth factor receptor ligand, a polymeric Ig receptor ligand, an erythropoietin receptor ligand, a purinoceptor ligand, and a metaloproteinase ligand.

110. The pharmaceutical of claim 106 wherein the bifunctional complex further comprises a linker.

111. An oncolytic composition comprising a carrier and a replicon that lacks a heterologous nucleic acid.

112. A method of treating an organism having a tumor comprising administering a pharmaceutically effective amount of replicons to the animal.
